(19) United States
(12) Patent Application Publication
(10) Pub. No.: US 2025/0204980 A1
(43) Pub. Date: Jun. 26, 2025

(54) SYSTEMS FOR TRAVERSING AND LACERATING A VALVE LEAFLET

(71) Applicant: Excision Medical, Exton, PA (US)

(72) Inventors: Greg Alan Walters, Exton, PA (US); Chad J. Smith, Phoenixville, PA (US); Mike A. Dotsey, Chester Springs, PA (US); Bryan Fitzgerald Allard, Royersford, PA (US); Francois Jouin, Malvern, PA (US); Shawn Sabu Cherian, Bridgeport, PA (US); Alison Mikayla Kane, Phoenixville, PA (US); Tessa Mazzerella, Chesterbrook, PA (US)

(73) Assignee: Excision Medical, Exton, PA (US)

(21) Appl. No.: 18/990,745

(22) Filed: Dec. 20, 2024

Related U.S. Application Data
(60) Provisional application No. 63/614,224, filed on Dec. 22, 2023.

(51) Int. Cl. A61B 18/14; A61B 18/00; A61B 18/16; A61M 25/01

(52) U.S. Cl. CPC A61B 18/1492; A61B 2018/00369; A61B 2018/00601; A61B 2018/1422; A61B 2018/144; A61B 2018/165; A61M 25/0105

(57) ABSTRACT

The present disclosure describes a surgical systems for traversing and lacerating a valve leaflet.

### CROSS-REFERENCE TO RELATED APPLICATION

The present application claims priority to and the benefit of U.S. Provisional Patent Application No. 63/614,224 filed Dec. 22, 2023, the contents of which is hereby incorporated in its entirety.

### TECHNICAL FIELD

The present disclosure relates to systems for excising and/or lacerating a portion of a native heart valve or an implanted heart valve.

### BACKGROUND

Transcatheter aortic valve replacement (TAVR) is an alternative option for the treatment of patients with severe calcific aortic stenosis. Indeed, TAVR may become the preferred therapy for all patients irrespective of surgical risk. However, transcatheter heart valves (THV) may fail in the future and repeat intervention may be required. So-called redo-transcatheter aortic valve implantation (TAVI) or TAVR may lead to risks of coronary obstruction due to the leaflet of the failed valve being pushed up by the new valve and leading to obstruction of blood flow to the coronary arteries. TAVR in failed surgical bioprostheses is common. However, TAVR in failed transcatheter bioprostheses (i.e., transcatheter heart valve-in-transcatheter heart valve) will also become increasingly common. In both situations there is a risk of coronary obstruction. The risk of coronary obstruction can be predicted with the use of cardiac computed tomography. If the predicted risk of coronary occlusion is high, then percutaneous valve-in-valve intervention may be prohibitive. In some cases, the cause of the coronary obstruction is related to the leaflets of the failed surgical or transcatheter heart valve that are pushed up and prevent flow of blood to the coronary arteries.

### SUMMARY

There is a need for systems, devices, and procedures for excision of portions of a native aortic valve and/or mitral valve or an implanted artificial aortic valve and/or mitral valve. An embodiment of the present disclosure includes a surgical system for excising portions of an aortic valve, a mitral valve, and artificial implanted valves.

### BRIEF DESCRIPTION OF THE DRAWINGS

The foregoing summary, as well as the following detailed description of illustrative embodiments of the present application, will be better understood when read in conjunction with the appended drawings. For purposes of illustrating the present application, the drawings show exemplary embodiments of the present disclosure. It should be understood, however, that the present disclosure is not limited to the precise arrangements and instrumentalities shown in the drawings. In the drawings:

FIG. 1 is a perspective view of a distal end of the surgical system according to an embodiment of the present disclosure;

FIG. 2 is a bottom view of the distal end of the surgical system illustrated in FIG. 1;

FIG. 3 is a sectional side view of the distal end of the surgical system illustrated in FIG. 2, taking along line I-I;

FIG. 4 is an exploded perspective view of a distal end of the surgical system shown in FIG. 1;

FIG. 5 is a perspective view of the cutting assemblies of the surgical system shown in FIG. 1;

FIG. 6 is an end view of the distal end of the surgical system shown in FIG. 1, with the cutting assemblies shown in FIG. 5 removed;

FIG. 7 is a perspective sectional view of the distal end of the surgical system shown in FIG. 1;

FIG. 8 is a perspective view of a cartridge assembly of the surgical system shown in FIG. 3;

FIG. 9 is a perspective view of a cutting hooks assembly of the surgical system shown in FIG. 1;

FIGS. 10-12 illustrate a cutting hook assembly according to another embodiment of the present disclosure;

FIGS. 13-15 illustrate a cutting hook assembly according to another embodiment of the present disclosure;

FIG. 16 is a perspective view of a distal end of a surgical system and a single cutting hook assembly in a side-by-side configuration, according to another embodiment of the present disclosure;

FIG. 17 is a perspective view of a distal end of a surgical system and a single cutting hook assembly according to another embodiment of the present disclosure;

FIG. 18 is a perspective view of a distal end of a surgical system and a single cutting hook assembly according to another embodiment of the present disclosure;

FIG. 19 is a perspective view of a distal end of a surgical system and a hook assembly according to another embodiment of the present disclosure;

FIG. 20 is a perspective view of a distal end of a surgical system and a hook assembly according to another embodiment of the present disclosure;

FIG. 21 is a perspective view of a distal end of a surgical system and a hook assembly according to another embodiment of the present disclosure;

FIG. 22 is a perspective view of a distal end of a surgical system according to another embodiment of the present disclosure, illustrating an insertion configuration;

FIG. 23 is a perspective view of a distal end of a surgical system according to another embodiment of the present disclosure, illustrating a cutting configuration, respectively;

FIG. 24 is a perspective view of a distal end of a surgical system according to another embodiment of the present disclosure, illustrating an insertion configuration;

FIG. 25 is a perspective view of a distal end of a surgical system according to another embodiment of the present disclosure, illustrating a cutting configuration;

FIG. 26 illustrates a steerable catheter assembly including a traversing catheter, a hook cutting catheter, and a steerable catheter of a surgical system according to another embodiment of the present disclosure;

FIGS. 27-29 illustrate a surgical system according to another embodiment of the present disclosure, showing a cutting assembly with a movable anchor for securing the cutting assembly in position;

FIGS. 30-32 illustrate a surgical system according to another embodiment of the present disclosure, showing a cutting assembly with a movable cutting hook and support arms for holding a leaflet of a valve;

FIGS. 33-34 illustrate a surgical system according to another embodiment of the present disclosure, showing a cutting assembly with a movable cutting hook without support arms;

FIG. 35 illustrates a surgical system according to another embodiment of the present disclosure, illustrating a steerable catheter assembly; and

FIG. 36 illustrates a cutting assembly of the surgical system according to another embodiment of the present disclosure.

### DETAILED DESCRIPTION OF ILLUSTRATIVE EMBODIMENTS

Systems and methods as set forth in the present disclosure may be used to access, traverse, and lacerate leaflets of a heart valve. In other cases, systems and methods may be used to remove portions of leaflets or other tissue or deposits from an aortic or mitral valve, whether native or implanted. As used herein, a cutting assembly may refer to one or more hooks, slit cutters, linear or curve cutting edges, which may or may not be configured to include electrodes to cut tissue. In some cases, the cutting assemblies may cut an opening or slit in the leaflet, in a first phase of operation, typically using a first electrode. Forming an initial slit or opening in the leaflet as described herein may be referred to as traversing the leaflet and is typically associated with advancing the system in a distal direction while applying electrical energy to the first electrode of the cutting assembly. In the second phase of operation, the cutting assembly, via a second electrode distinct from the first electrode, lacerates the leaflet by applying tension force and/or retracting the cutting assembly while electrifying another part of the cutting assembly.

The surgical systems as described herein are used to facilitate the cutting and/or removal of leaflet portions from a valve of a heart. The surgical system can be used to access and/or excise aortic valves, such as native valves or implanted artificial valves. The surgical system as described herein may be used in combination with a TAVR sheath (not shown), used to access the valve via the descending aorta and into the ascending aorta toward the valve structure typically located inferior to or near the coronary ostia. While a system may be used with TAVR, embodiments of the present disclosure may be used with surgical or native valves as well. In embodiments of the present disclosure, an aortic arch may include exemplary TAVR valve (not shown) implanted at the aortic annulus and having damaged leaflets that fail to coapt. Certain systems as described herein can be used to excise or lacerate damaged or calcified leaflets.

The surgical system 10 (and related methods) as described herein are configured to provide access to and safely remove a portion of the valve structure. The surgical system 10 may therefore include one or more distinct elements designed to guide the system toward and then traverse, cut, and remove a portion of the leaflet of the valve. More specifically, the surgical system 10 may include one or more of the following elements, cither combined in a single assembly or comprising separate modular components, designed to achieve desired leaflet or tissue removal: (a) a steering element for targeting the system toward the desired tissue site; and/or (b) one or more cutting assemblies 30 in the form of hooks, tips, slit cutters, and the like. In some instances, the surgical system 10 may include anchors to secure the surgical system in place to add in excision and/or laceration of the tissue, e.g. the leaflet of the valve. The surgical system 10 may include a handle (not shown) and may include one or more actuators (not shown) that are configured to control operation of and relative movement of elements of surgical system 10 as described herein.

The surgical system 10 is generally sized and configured for insertion into a TAVR sheath positioned in the ascending aorta. The system 10 may include additional devices, such as guide wires, introducers, etc., to facilitate introduction of the surgical system into the aortic arch. In terms of size, the distal end and shafts of the system may be sized to fit within a TAVR sheath. For example, the surgical system shaft 12 may have an outer diameter, measured perpendicular to a central axis 1 thereof, up to about 14 F. The inner diameter of the introducer (if used) is sized to fit around a guidewire and that may be at most 0.035 inches. However, the inner diameter of the introducer or other components, which could receive a guide wire, may vary. Furthermore, the effective length of the surgical system, such as the portion that extends from the entry site of the patient to the target location in the heart may vary. In some examples, the effective length may range between about 40 cm up to about 150 cm, and any intervals therebetween. Accordingly, the surgical system size and configuration could vary as needed.

The surgical system 10 includes elongated shafts that engage or are coupled to the handle (not shown) and are designed to extend to the target valve, either alone, through a steerable catheter, and/or through a procedural sheath, such as a TAVR sheath. Some or all of the elongated shafts as described herein may be elongated elements that are generally flexible and insertable into the TAVR sheaths. The shafts as described herein may be in the form of catheters, which include an internal channel through which other devices and elements or may pass. Its form as a catheter is not strictly required but would be useful, as needed, when coupled with other surgical devices for access to and engagement with a valve (implanted or native) in the aorta.

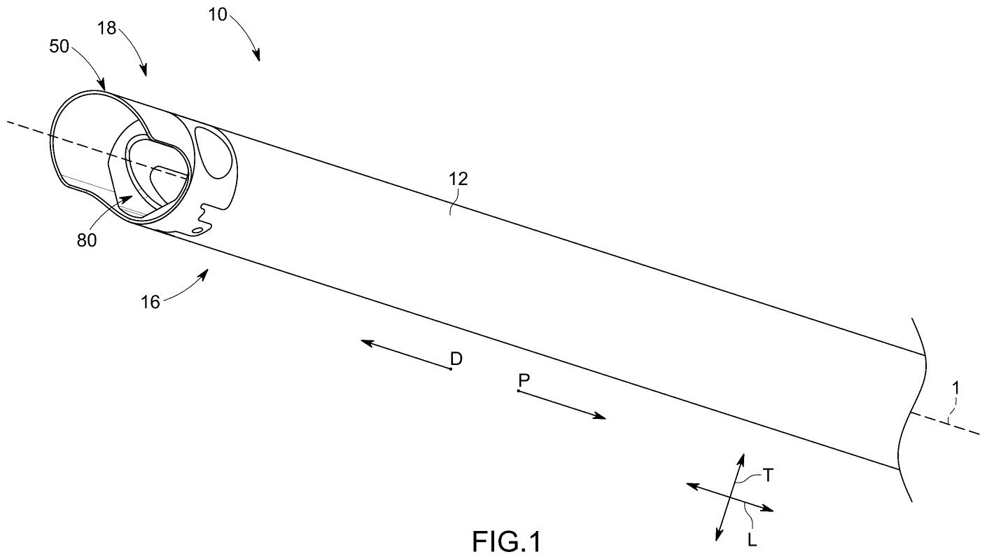

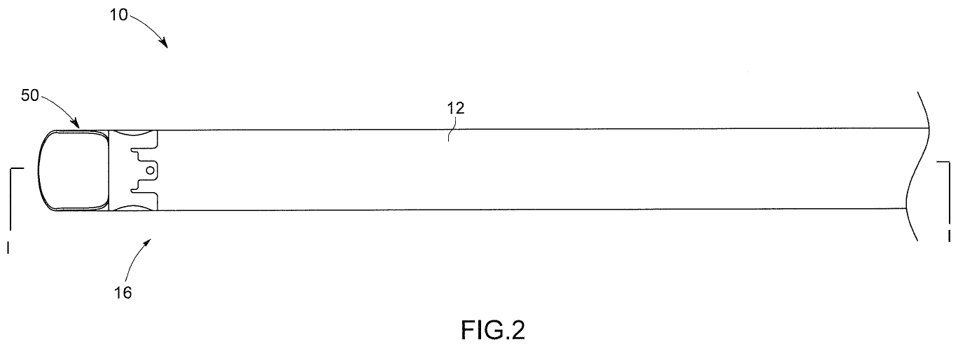

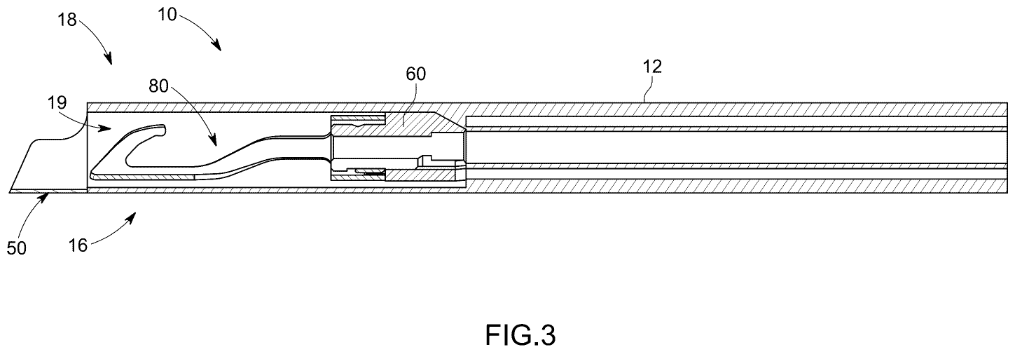

As illustrated in FIGS. 1-4, the elongate shafts 12 of the surgical system 10 includes a distal end 18 and a proximal end (not shown) that are spaced apart from each other along a central axis 1. The system 10, and the elongated shafts, are elongated along the central axis 1 which extends along a longitudinal direction L. Generally, a direction from the proximal end toward the distal end is referred to herein as the distal direction D. A direction from the distal end toward the proximal end is referred to herein as the proximal direction P (opposite the distal direction). A transverse direction T as used herein refers to a direction that crosses and is perpendicular or otherwise angled relative to the longitudinal direction L.

The surgical system 10 may include a steering element (not numbered). The steering element may be designed to be inserted through the TAVR sheath and target the distal end 18 of the system 10 toward the valve structure. The steering element may include a shaft with a distal end, a proximal end spaced from the distal end in a proximal direction along a central axis 1. In an alternative embodiment, the steering element, when in the form of a catheter, may also include an inner channel that extends between the distal and proximal ends thereof. A catheter is not strictly required to effectuate steering or targeting as described herein. The steering element is configured to present the distal end 18 of the system into the ascending aorta proximate the valve so that the cutting elements of the cutting assembly 30 can be actuated as needed to accomplish their respective functional objectives. In such an example, the distal end of the steering element, for example the distal end of shaft, can be steered or guided into position as needed to present the elements proximate the valve.

The cutting assemblies 30 form a part of the distal-most end of the shaft or are carried by the shaft. However, the cutting assemblies 30 may be disposed along a separate shaft or a catheter and inserted or moveable through and relative to the shaft. Like other elements described in this present application, the cutting element 50 may not be carried by a catheter per se. However, a shaft as used herein can include a catheter as described elsewhere in the present disclosure. The separate outer shaft may be configured to aid in the delivery and/or containment of a non-ionic solution, such as dextrose, to displace blood during electrification and concentrate electrical energy on the target leaflet.

The described elements are shown as separate components, one for creating a curved cut in the leaflet and one for excising a portion of the leaflet. However, it is possible that the elements described herein may be combined into a single surgical system that target, pierce, cut, and/or remove a leaflet. In one example, the surgical system may be inserted into the implanted TAVR sheath as a single unit. In such an example, the handle of the system is configured to facilitate the control of the various components and subcomponents, via actuators and the like, as described above.

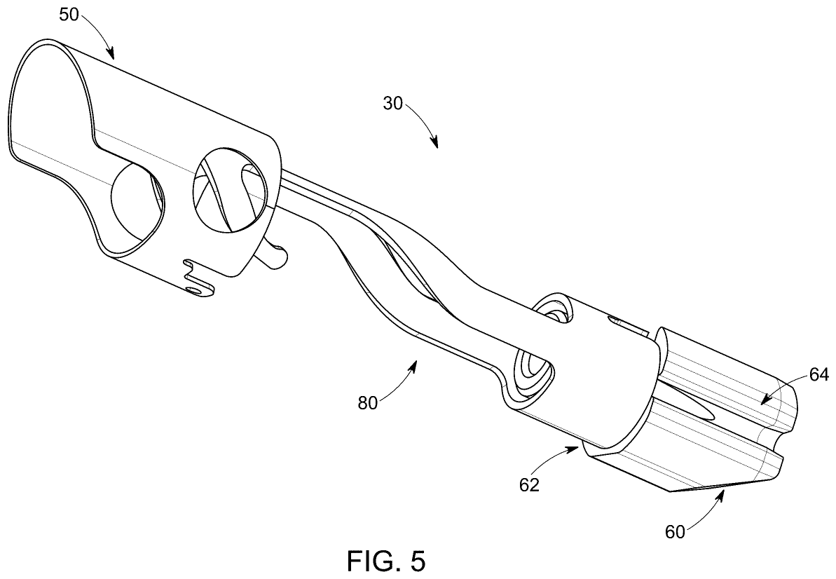

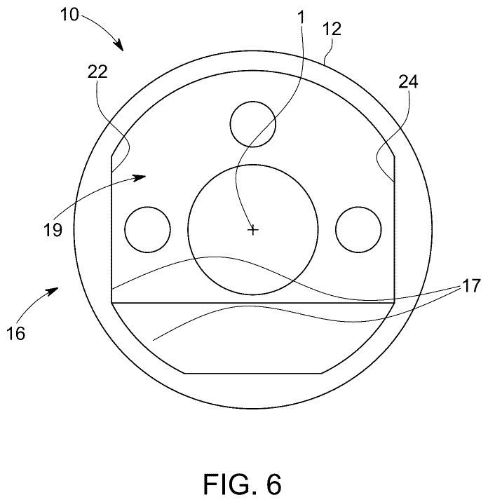

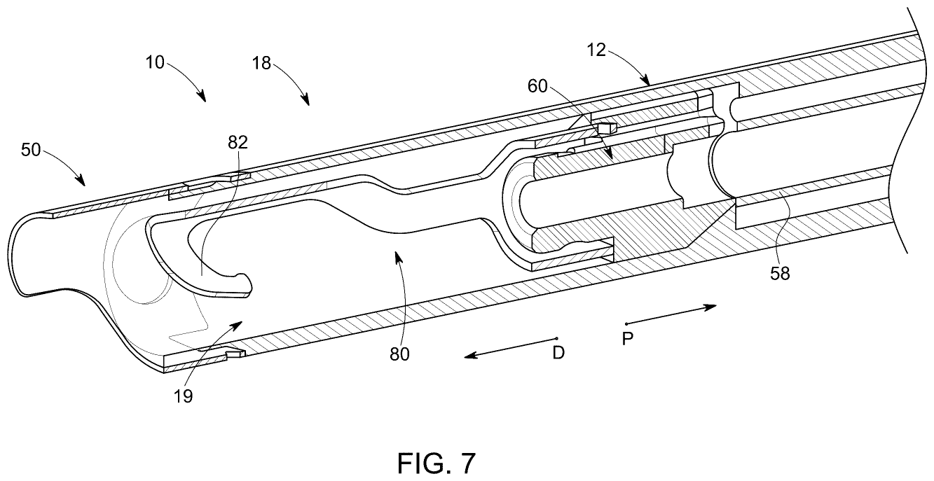

Continuing with FIGS. 1-9, the surgical system 10 includes an assembly configured to ensure alignment of the cutting hooks 80 relative a cutting element 50 in use. As shown the system 10 includes a shaft (or catheter) 12 having a proximal end (not shown), a distal end 16 opposite the proximal end along a central axis 1, an inner surface, and an inner channel 19 defined by the inner surface with the inner channel 19 extending from the distal end toward the proximal end of the catheter. The cutting assembly 30 includes a first cutting element 50 fixed to the distal end of the shaft, and a cartridge 60 located in the inner channel 19. A second cutting element (hook assembly) 80 is fixed to the cartridge 60.

The cutting element 50 is configured as a C-shape cutting element and extends around a portion of the central axis 1 of the catheter and defines a curved cutting edge. As shown, the cutting element 50 may have a C-shape to form a curved opening in the leaflet. The curved cutting edge of the cutting element 50 is a sharp element. In another example, the curved cutting edge is an electrode that is responsive to electrical energy supplied by a separate electrosurgical unit. In this description, electrical energy and RF energy are used interchangeably.

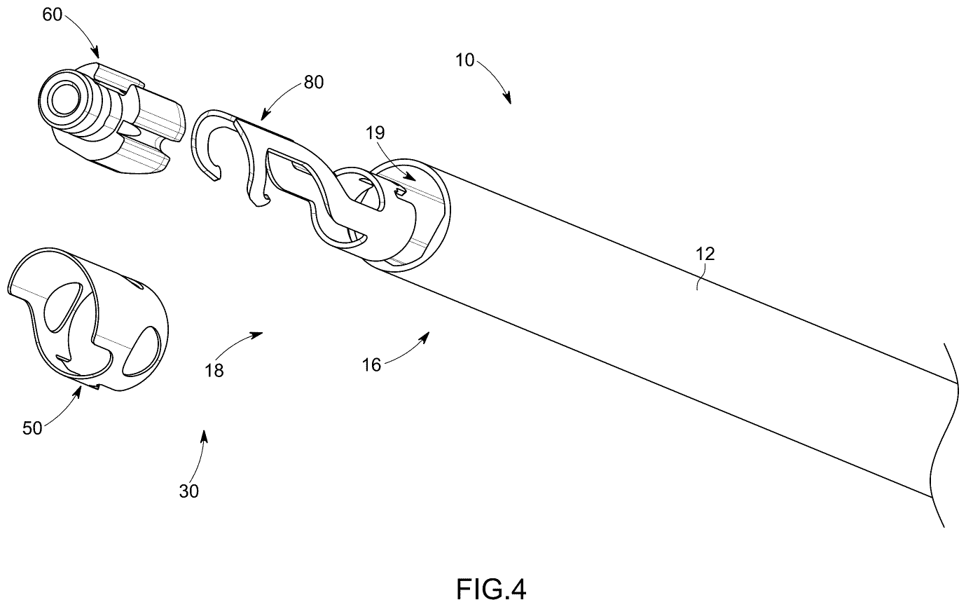

As shown in FIGS. 4-8, the cartridge 60 is keyed to an inner surface of the catheter/shaft 12 to prevent its rotation in the catheter/shaft. More specifically, the inner surface 17 of the catheter has a first flat portion 22 and a second flat portion 24 that is opposite the first flat portion such that the central axis 1 is between first flat portion 22 and the second flat portion 24. The cartridge 60 has a first side 62 that faces the first flat portion 22 and a second side 64 opposite the first side that faces the second flat portion 24. Configured this way, the cartridge 60 may move in a proximal direction P or a distal direction D in the catheter but will not rotate within the catheter. Also included is an inner shaft 58 having a distal end coupled to the cartridge 60. The inner shaft 58 is configured to cause the cartridge 60 to move in a distal direction or a proximal direction to advance the hook assembly 80 relative to the cutting element 50 and retract the hook assembly relative to the cutting element 50, respectively.

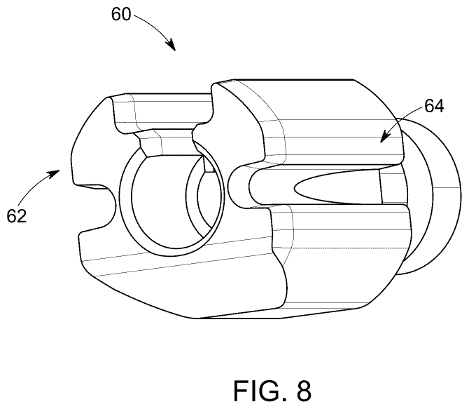

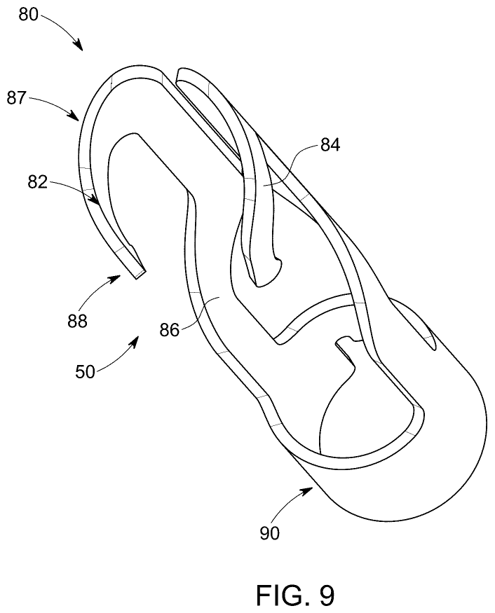

Referring to FIG. 9, the hook assembly 80 has a first hook 82 and a second hook 84. Each hook 82, 84 may include a shank 86, a bend 87, and a point or tip 88 that extends from the bend. The shank 86 of each hook is coupled to a hook base 90 that is fixed to the cartridge 60. The hooks are thus oriented to align with the cutting element 50 such that both the hooks and the cutting element 50 extend around a portion of the central axis 1. Constructed as described above, the hook assembly 80 and cartridge 60 are movable along a longitudinal direction but are not rotatable in the inner channel of the catheter 12. This, in turn, maintains proper alignment of the hooks 82, 84 relative to the cutting element 50 during use. The first hook 82 and second hook 84 may be configured as electrodes coupled to an RF unit (not shown). The hooks 82, 84 when configured as electrodes may be made of stainless steel, nitinol, or similar conductive material suitable for insertion in the aorta. Furthermore, the hooks 82, 84 may also include an insulative coating with denuded surfaces, such as at an inner surface of the bend of each hook 82, 84. Both insulated and denuded surfaces may ensure that energy is directed to the correct places in use when the hook assembly 80 is electrified for laceration.

In use, the user may advance the catheter into position in the aorta. The distal end 16 of the catheter 12 may be steered into the correct position using an actuator or control element (not shown or numbered) at the handle of the system 10. The leaflet can be pierced by a piercing element (not shown or numbered) and captured by a capture element (not shown or numbered) prior to the cutting steps. The cutting element 50 can then traverse the leaflet, forming a curve opening therein. Then, the user can advance the inner shaft 58 and cartridge 60 in a distal direction D so that the hook assembly 80 exits the catheter and extends distally beyond the cutting element 50 and through the curved opening in the leaflet. The first and second hooks 82, 84 may then be electrified via the RF unit (not shown) and the catheter retracted so that the first and second hooks lacerate the leaflet. The first and second hooks 82, 84 can splay apart, excising a portion of the leaflet in the process. The excised portion of the leaflet can then be removed from the surgical site. This can be repeated for each leaflet. Thereafter, the user can perform any needed addition procedure, such as inserting a new valve in the aorta.

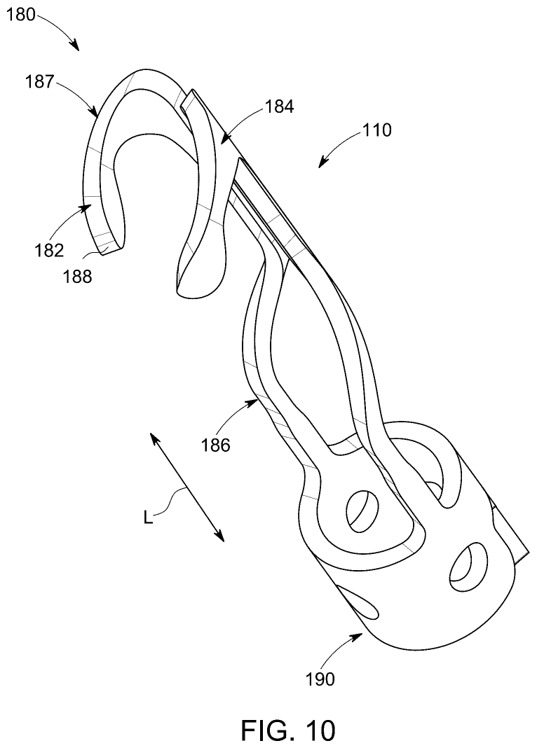

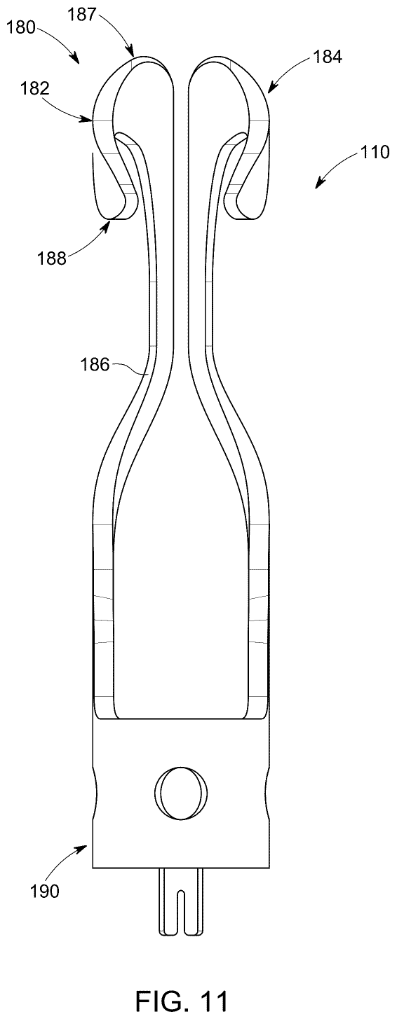

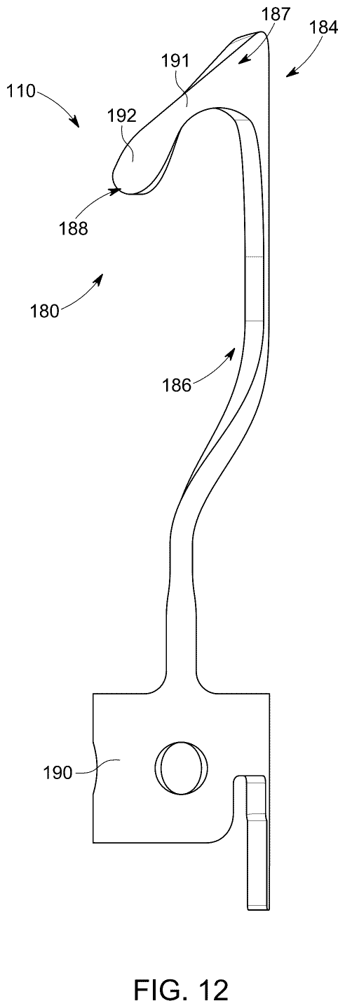

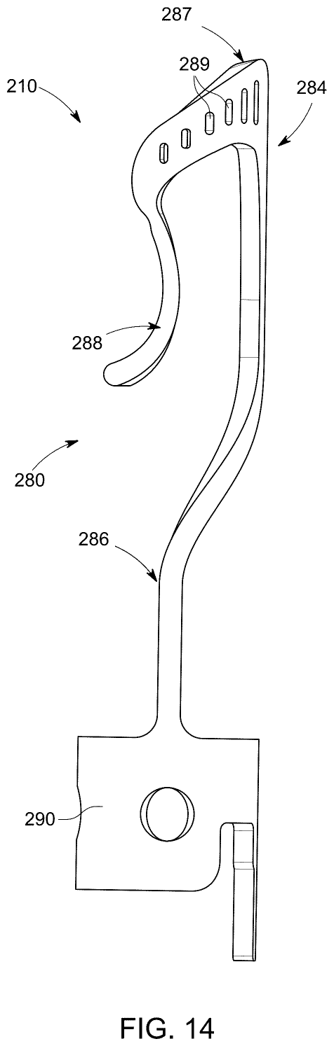

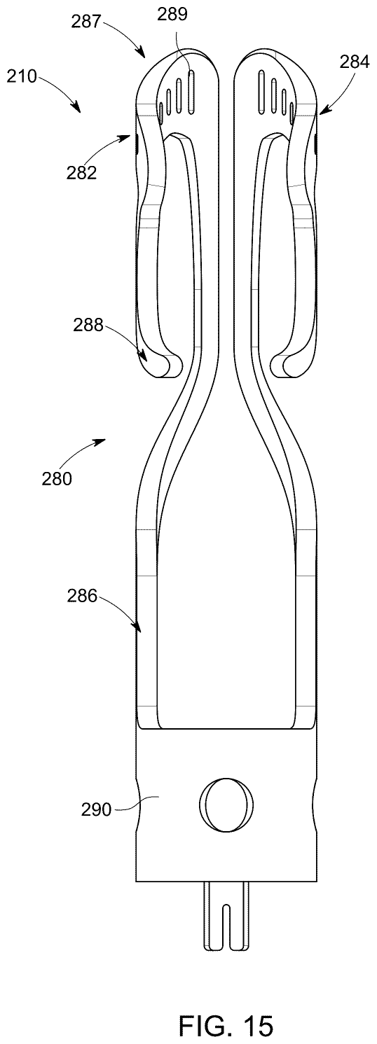

FIGS. 10-15 illustrate various hook assemblies used in the surgical systems for excising a portion of a leaflet, as described herein. The anatomy and configuration of the hook assemblies described, however, may be utilized in embodiments comprising single hook assemblies for lacerating a portion of a leaflet. In the illustrated embodiment, the configuration of the hook assemblies in the surgical systems described herein enable the hooks to be captured and deployed from the distal end of a catheter. The surgical systems 110, 210 include hook assemblies 180, 280 shown in FIGS. 10-15 include a shaft 112, 212 (not shown) that is elongated along a central axis 1 and has a proximal end 114, 214 (not shown) and a distal end 116, 216 (not shown) opposite the proximal end along the central axis 1.

As shown in FIGS. 10-12, the system 110 includes a hook assembly 180 carried by the distal end of the shaft 112 via a base 190. The hook assembly 180 may have one or more hooks. In the embodiment shown, the hook assembly 180 has a first hook 182 and a second hook 184 although one hook could be used. Each hook includes a shank 186, a bend 187, and a tip 188 extending from the bend 187. The hook assembly 180 may include a base 190 coupled to the shank(s) that is fixed to the shaft 112.

The shank 186 itself has a proximal end and a distal end, and a curved portion between the proximal end of the shank 186 and the distal end of the shank 186. The elongated portion of the tip 188 has a narrow portion 191 and a lobe 192 that defines a terminal end of the tip 188. As shown in FIGS. 10-12, the tip 188 is angled with respect to the distal end of the shank 186 and the bend 187. The tip 188 also curves around a part of the central axis 1 of the shaft 112. The configuration of the tip 188 of the hook assemblies in the surgical systems described herein allows the tip to remain engaged with the tissue while cutting.

When the hook assembly 180 has a first hook 182 and a second hook 184, they are configured to be mirror images of each other. In this manner, the tips 188 of the hooks 182, 184 curve around the axis toward each other, but do not always contact to define a gap therebetween. Furthermore, in the embodiment shown, the tips 188 are configured to prevent significant flexing relative to the bend 187 and shank 186. However, as described in other embodiments, the tips 188 of the first hook and the second hook may be configured to flex during use.

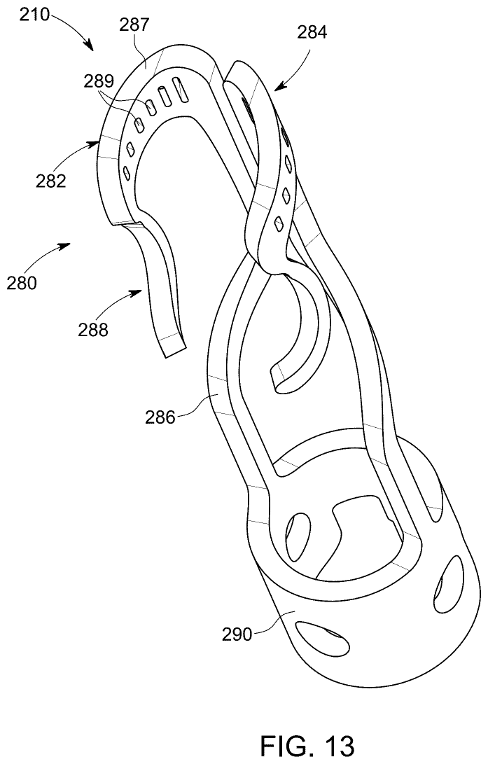

Referring to FIGS. 13-15, another embodiment of the surgical system 210 with a hook assembly 280 is shown (other components, such as the shaft are not shown). However, the surgical systems as described herein may include the shaft, cartridge and alignment features as described above. In this embodiment, the hook assembly 280 includes one or more hooks, e.g. a first hook 282 and a second hook 284. Each hook includes a shank 286, a bend 287, a tip 288, and a base 290. The tip 288 includes an elongated portion that extends in a proximal direction and curves or is angled in a direction away from the shank 286. In one embodiment, as shown FIGS. 13-15, the bend 287 and/or portion of the tip may include one or more apertures 289. The apertures 289 may be used to dissipate heat. However, other embodiments may not include one or more apertures 289. When the hook assembly 280 has a first hook 282 and a second hook 284, they are configured to be mirror images of each other. In this manner, the tips 288 and bends 287 of the hooks curve around the axis toward each other, but do not always contact to define a gap therebetween. Furthermore, the bend 287 of the hook defines an inner surface that may be or include an electrode. Configured this way, each hook includes a high strain zone and a high heat zone that is separate from the high strain zone. The high strain zone may be located at or near where the tip 288 and bend 287 meet while the high heat zone is located at the inner surface of the bend 287 where the electrode is located. In some examples, the hook has an insulative coating, which is denuded at the inner surface of the bend to form the electrode and site of laceration. In use, this allows the tip to easily flex when retracted and lacerating the leaflet, while the high heat zone focuses the RF energy at the point where bend contacts the leaflet, focusing the energy to lacerate. By creating a high strain zone, which is flexible, separate from the electrode, which generates heat, hook structure and integrity is preserved during use.

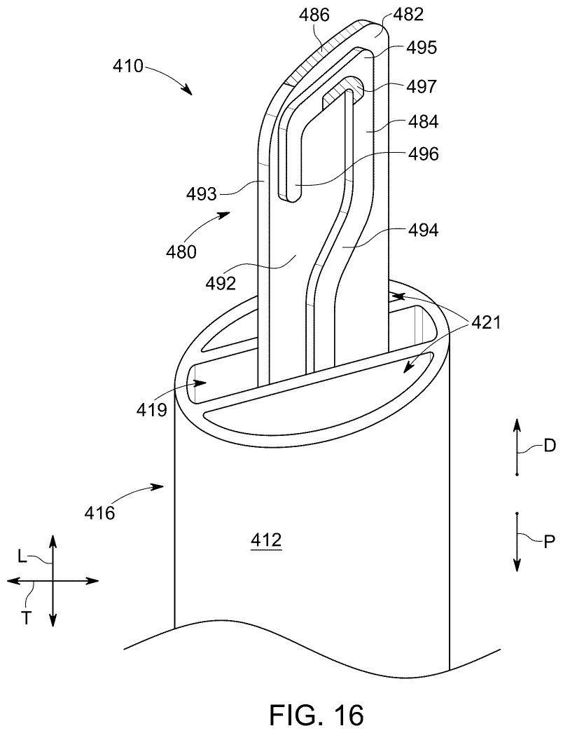

FIG. 16 illustrates another embodiment of a surgical system 410 including a cutting assembly 480 carried by a shaft 412, e.g. a catheter for lacerating a portion of a leaflet. In this example, the shaft 412 has a proximal end (not shown), a distal end 414 opposite the proximal end, and a channel 419 that extends from the distal end toward the proximal end. The cutting assembly 480 may be movably located in the channel 419. However, the shaft 412 may have additional channels 421 or lumens that can be used to introduce various other elements or components to the surgical site during use. These are intended to a) allow for flushing or priming the system prior to introduction to the patient, b) allow removal of emboli, such as air and other debris after cutting, and throughout, c) provide for hemodynamic monitoring of the blood pressure, d) allow for contrast injection, as needed, and/or e) permit the delivery of a non-ionic solution, such as dextrose, to displace blood during electrification and concentrate electrical energy on the target leaflet.

Continuing with FIG. 16, the cutting assembly 480 includes a slit cutter 482 configured to form a slit in a tissue, being movable in the channel, and a hook 484 adjacent to and slidable relative to the slit cutter. The slit cutter and the hook are independently movable relative to each other. Alternatively, the hook may be spring loaded to move forward and engage the leaflet when the slit cutter is retracted.

The slit cutter 482 includes a distal cutting surface 486 configured to cut through the leaflet. As shown, the slit cutter 482 may be coated with an insulative coating and have a denuded distal cutting surface. Alternatively, a separate electrode may be affixed to the distal end of the slit cutter 482. In the example shown in FIG. 16, the slit cutter 482 includes a planar cutting body having a first planar surface 490, a second planer surface 492 opposite the first planar surface 490, and sidewalls 493 that extends between the first planer surface 490 and the second planar surface. In this case, the distal-most portion of the slit cutter 482 defines the distal cutting surface 486.

Continuing with FIG. 16, a hook 484 may be located in the channel and is movable relative to the slit cutter 482. As shown, the hook 484 has a shank 494, a bend 495, a tip 496, and an inner cutting surface 497 at an inner surface of the bend 495. Configured this way, the distal cutting surface 486 and the inner cutting surface 497 are oriented in generally opposite directions so that advancing of the slit cutter 482 forms a slit while retracting of the hook 484 lacerates the leaflet during use. As shown, the hook 484 may be coated with an insulative coating and denuded at the inner surface of the bend to define the inner cutting surface 497. Accordingly, the inner cutting surface can be an electrode. Alternatively, a separate electrode may be affixed to the inner surface of the bend of the hook 484.

The cutting assembly 480 can operate using RF energy. Thus, the distal cutting surface of the slit cutter 486 may be an electrode and the inner surface of the hook bend 497 may be an electrode. As shown, the slit cutter 482 and the hook 484 electrodes may be electrically isolated from each other by, for example, their respective insulative coatings. Furthermore, the slit cutter 482 and the hook 484 electrodes may be independently coupled to electric power. Accordingly, embodiments of the present disclosure include a cutting assembly with a first electrode and a second electrode separate from the first electrode. The first and second electrodes can be oriented in different directions so that advance in one direction cause cutting or traversing of a tissue (e.g. the leaflet) and movement of the cutting assembly 480 in the opposite direction to cause laceration of the tissue (e.g. the leaflet). As depicted in FIG. 16, the leading end of the slit cutter 482 may be positioned slightly distal to the leading end of the hook 484 to optimize electrical contact between the distal cutting surface of the slit cutter and the target leaflet while slitting the leaflet.

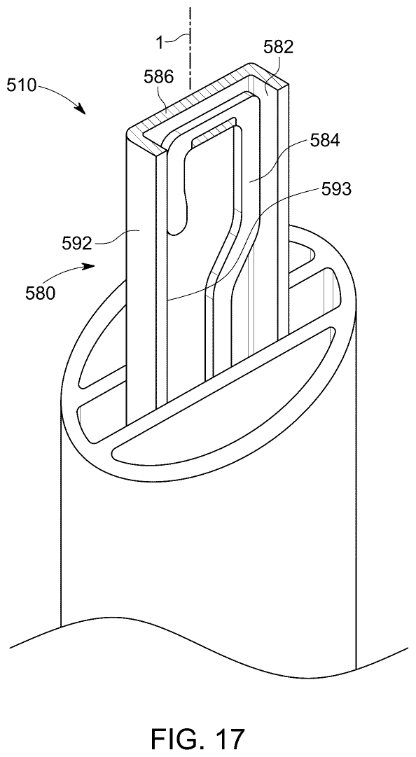

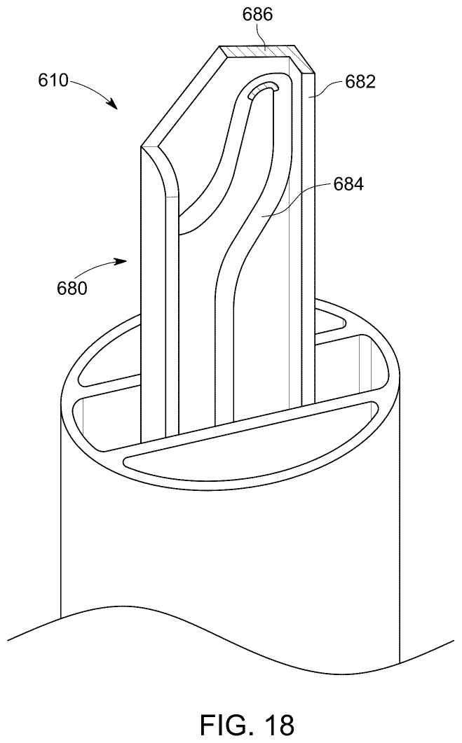

FIGS. 17 and 18 illustrate other embodiments of a surgical system 510, 610 including cutting assemblies 580, 680 for lacerating a portion of a leaflet. The cutting assembly 580, 680 as shown in FIGS. 17 and 18 are substantially similar to the cutting assembly shown in FIG. 16, except that the slit cutter 582, 682 is configured differently. More specifically, as shown in FIG. 17, the slit cutter 582 includes a distal cutting surface 586 configured to cut through the leaflet. And the slit cutter 582 includes a wall that extends partially around a central axis to form a guide channel. This configuration ensures easy passage of the slit cutter 582, 682 and hook 584, 684 through the leaflet after conducting the slit cut. As shown, the wall has an outer surface 592, an inner surface 593, and the distal cutting surface 586. Here, the hook is nested within the guide channel. Like the embodiment shown above, the slit cutter 582 may include an insulative coating with a portion at the distal surface denuded to from a distal cutting surface. Alternatively, separate electrodes may be affixed to the distal end of the slit cutters 582, 682. In FIG. 18, the distal cutting surface 686 of the slit cutter 682 is further tapered to isolate the distal cutting surface further. This configuration reduces the electrode size (i.e. denuded surface) to increase current density. The hooks 584, 684 shown in FIGS. 17 and 18 are substantially similar to hook 484 shown in FIG. 16. The leading end of the slit cutter 582, 682 may be positioned slightly distal to the leading end of the hook 584, 684 to optimize electrical contact between the distal cutting surface of the slit cutter and the target leaflet while slitting the leaflet.

Continuing with FIGS. 16-18, in use, the method may include positioning a distal end of a shaft toward a leaflet of a valve with the shaft having a channel, a slit cutter and a hook each located in the channel. A user can electrify the distal cutting surface of the slit cutter to traverse the leaflet. Then, the user can advance the slit cutter and a hook through the leaflet so that the slit cutter forms a slit in the leaflet. Next, a user may retract the slit cutter relative to the hook and engage the hook with the leaflet. Electrical energy is then supplied to the hook, and to the inner surface of the bend in particular to lacerate the leaflet. Further laceration is facilitated by retracting the hook.

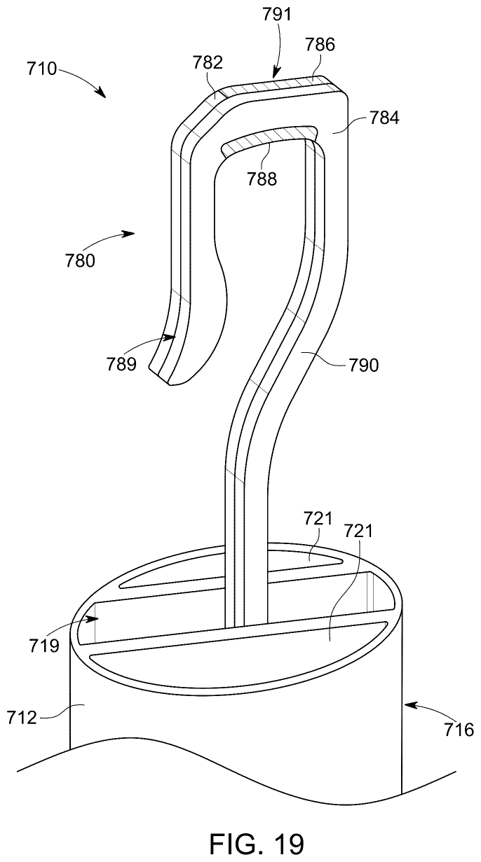

FIGS. 19-21 illustrate another embodiment of a surgical system 710 used to lacerate a leaflet of a valve. As shown in FIG. 19, the surgical system 710 includes a shaft having a proximal end (not shown), a distal end 716 opposite the proximal end, and a channel 719 that extends from the distal end 716 toward the proximal end. The surgical system 710 further includes a cutting assembly 780 having a first hook 782 and a second hook 784 with cutting surfaces 786, 788 that are orientated in generally opposite directions to facilitate traverse through the leaflet and lacerating the leaflet during use.

The shaft 712 may include additional channels 721 or lumens to a) allow for flushing or priming the system prior to introduction to the patient, b) allow removal of emboli, such as air and other debris after cutting, and throughout, c) provide for hemodynamic monitoring of the blood pressure, d) allow for contrast injection, as needed, and/or c) permit the delivery of a non-ionic solution, such as dextrose, to displace blood during electrification and concentrate electrical energy on the target leaflet.

Continuing with FIG. 19, the cutting assembly 780 of the system 710 includes a first hook 782 and a second hook 784. As shown, the first hook 782 has a shank 790, a bend 791, and a distal cutting surface 786 at an outer portion of the bend 791. A second hook 784, also in the channel and arranged adjacent the first hook 782, includes a shank 790, a bend 791, and an inner cutting surface 788 at an inner portion of the bend 791. Configured this way, the distal cutting surface 786 of the first hook 782 and the inner cutting surface 788 of the second hook 784 are oriented in opposite directions. As shown, the tips 789 of the first and second hooks 782, 784 have a portion that curves away from the shank 790 and forms a gape between the tip and the shank. In one configuration, the area bounded by the bend 791 and proximal portion of the shank 790 has a distance that is generally greater than the distance of the gape between the tip and the shank.

Continuing with FIG. 19, the first and second hooks 782, 784 are movable together relative to the shaft. In this way the first hook 782 is configured to form a slit or cut in a leaflet of a valve, and the second hook 784 is configured to lacerate the leaflet of the valve dependent on the movement directions of the cutting assembly. More specifically, movement of the first hook 782 and the second hook 784 in a distal direction may cause the first hook 782 to form a slit in a leaflet of a valve, and movement of the first hook 782 and the second hook 784 in the proximal direction that is opposite the distal direction causes the second hook 784 to lacerate the leaflet of the valve. The first hook 782 and the second hook 784 are attached to each other. For example, the first hook 782 and second hook 784 may be coupled together with a sleeve, wrap or coating that keeps the first and second hooks adjacent and moving together. The two hooks are electrically isolated from each other by, for example, their respective insulative coatings. The leading surface 786 of the first hook 782 may be positioned distal to the leading surface of the second hook 784 to optimize electrical contact between the distal cutting surface of the first hook and the target leaflet while slitting the leaflet. Likewise, the inner cutting surface 788 of the second hook 784 may be positioned proximal to the inner surface of the first hook 782 to optimize electrical contact between the inner cutting surface of the second hook and the target leaflet while lacerating the leaflet.

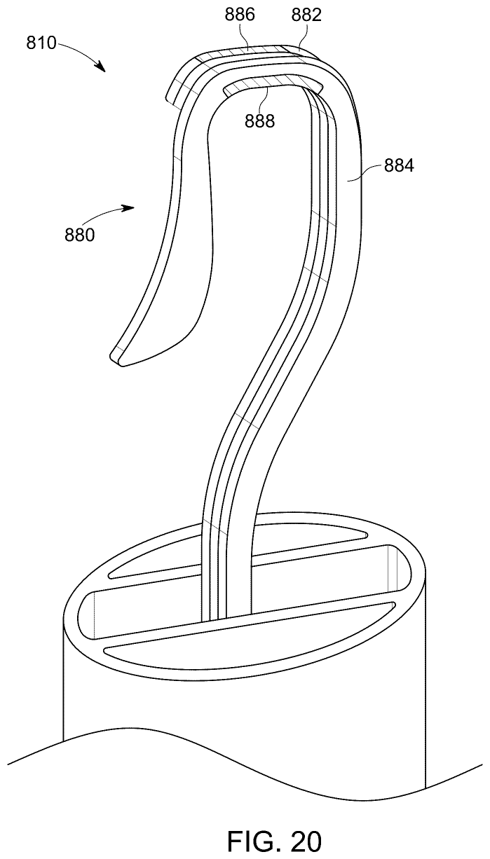

Turning to FIG. 20, another embodiment of the surgical system 810 is shown having a different configured cutting assembly 880. As shown, the first hook 882 includes only the bend and does not have a tip that extends downwardly in the proximal direction, contrasted with the first hook 782 of the cutting assembly shown in FIG. 19. The second hook 884, however, has a tip that is configured similar to the second hook of the cutting assembly shown in FIG. 19. However, the first and second hooks of the cutting assembly 880 have similar features to the cutting assembly 780 shown in FIG. 19 and described above. For instance, the first hook 882 has a distal cutting surface 886 at an outer portion of the bend and the second hook 884 has an inner cutting surface 888 at an inner portion of the bend. Again, configured this way, the distal cutting surface 886 of the first hook 882 and the inner cutting surface 888 of the second hook 884 are arranged in generally opposite directions.

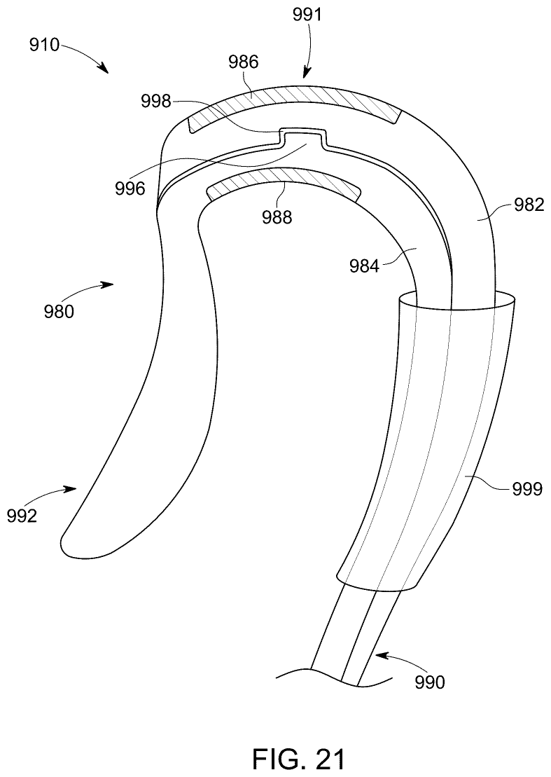

In FIG. 21, another embodiment of the surgical system 910 and cutting assembly 980 for lacerating a leaflet is shown. The cutting assembly 980 has a first hook 982 with a bend and distal cutting surface 986 on the bend. The second hook 984 has a shank 990, a bend 991, and a tip 992 with an inner cutting surface 988 at an inner portion of the bend. As shown, however, the first hook 982 is positioned distal relative to the second hook 984. Furthermore, the second hook 984 is nested in the bend of the first hook 982. This is accomplished via a first engagement member 996, in the form of a recess at the inner surface of the bend of the first hook, and a second engagement member 998, which may be a projection that is located on a distal surface at the bend of the second hook. In this way, the first and second engagement members 996, 998 mate so that the first and second hooks are coupled together and do not slide apart. An additional attachment member, shown as a sleeve 999 is used to maintain the position of the hooks 982, 984 relative to each other. While the engagement members are shown at the bends of each hook, the engagement members could be located along the shank or other portions of the hooks.

The cutting surfaces of the first hook 982 and the second hook 984 may be configured as electrodes, thereby forming a cutting assembly 980 that has first and second electrodes. In this example, the first and second hooks 982, 984 may be electrically isolated from each other by, for example, their respective insulative coatings. In addition, the first and second hooks 982, 984 are independently coupled to electric power. Further, the first and second hooks 982, 984 are insulated and include a first denuded portion at the distal cutting surface 986 of the first hook 982 and a second denuded portion 988 at the inner cutting surface of the second hook 984. Alternatively, separate electrodes may be affixed to the distal end of the first hook 982 and the inner cutting surface of the second hook 984. RF power may be supplied to the respective cutting surfaces, in sequence to effectuate cutting of the tissue in the desired sequence during the surgical procedure.

In another embodiment, the surgical system 710, 810, 910 may include the shaft as described above and a cutting assembly. The cutting assembly may include a single hook carried by the shaft. In this example, the hook has a shank, a bend, a tip and a distal electrode at an outer portion of the bend, and an inner electrode at an inner surface of the bend. In this way, the single hook is configured to cut tissue when advancing in both a distal direction and a proximal direction.

In use, the surgical system described above and shown in FIGS. 19 through 21 may include, initially, positioning a distal end of a shaft toward a leaflet of a valve. The user may electrify a distal cutting surface of the first hook to form a slit in a leaflet of a valve. The user can, via one or more actuators or control elements, advance the first hook and a second hook through the slit in the leaflet in a distal direction. At this point, the user can engage the first and second hooks with the leaflet. Then, the inner cutting surface, or electrode, may be electrified. While electrifying the inner cutting surface of the second hook, the user can retract the cutting assembly in a proximal direction to lacerate the leaflet with the inner cutting surface of the second hook. As described above, this sequence can be also achieved with a single hook comprising two electrodes.

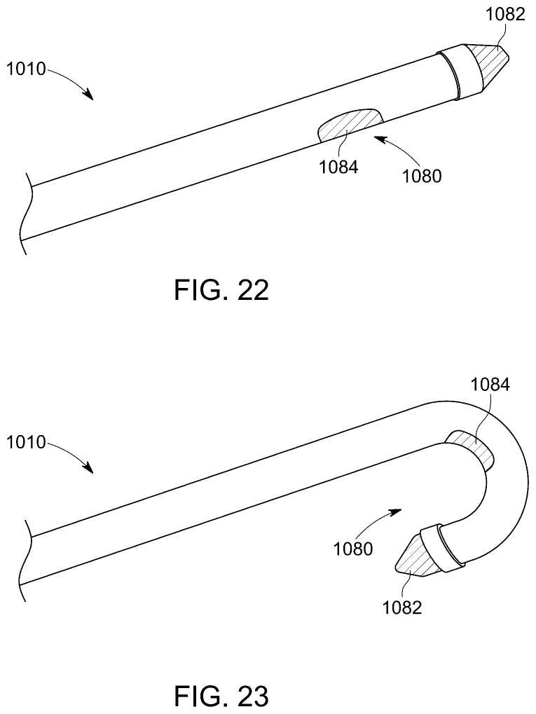

Turning to FIGS. 22 and 23, another surgical system 1010 having a cutting assembly 1080 are shown configured with at least two electrodes for traversing the leaflet and lacerating the leaflet. In this embodiment, the surgical system 1010 has a proximal end, a distal end opposite the proximal end, and a channel that extends from the distal end toward the proximal end. The system includes a first electrode 1082 located at a distal-most end of the distal end of the shaft, and a second electrode 1084 located along a sidewall of the shaft at a location that is proximal relative to the first electrode. Included is an internal pull wire in the shaft having a distal end that is coupled to the distal end of the shaft. The internal pull wire configured to, upon application of tension to the internal pull wire, cause the distal end of the shaft to curve into a hook shape such that the second electrode 1084 is positioned on an inner surface of a bend in the hook. Thus, in this manner the surgical system 1010 has a traversing configuration where the shaft is generally linear, as shown in FIG. 22, and a lacerating configuration wherein the distal end of the shaft is curved in the form of the hook, as shown in FIG. 23. As with other embodiment of the present disclosure, the first and second electrodes 1082, 1084 are electrically isolated from each other. In addition, the first and second electrode 1082, 1084 are independently coupled to electric power. In this embodiment also, the system 1010 includes a steerable catheter having a channel extending therethrough. The shaft is movable in the channel of the steerable catheter to permit the distal end to extend out of a distal end of the steerable catheter.

In use, the surgical system described above and shown in FIGS. 22 and 23 may include, initially, positioning a distal end of a shaft toward a leaflet of a valve. The user may activate the first electrode to traverse the leaflet in a distal direction. After traversing the leaflet, the user may actuate the system from the traversing configuration to the lacerating configuration, such that the distal end of the shaft is curved in the form of a hook. At this point, the user can engage the hook with the leaflet. Then, the inner cutting surface, or second electrode, may be electrified. The user can retract the cutting assembly in a proximal direction to lacerate the leaflet with the inner cutting surface of the hook.

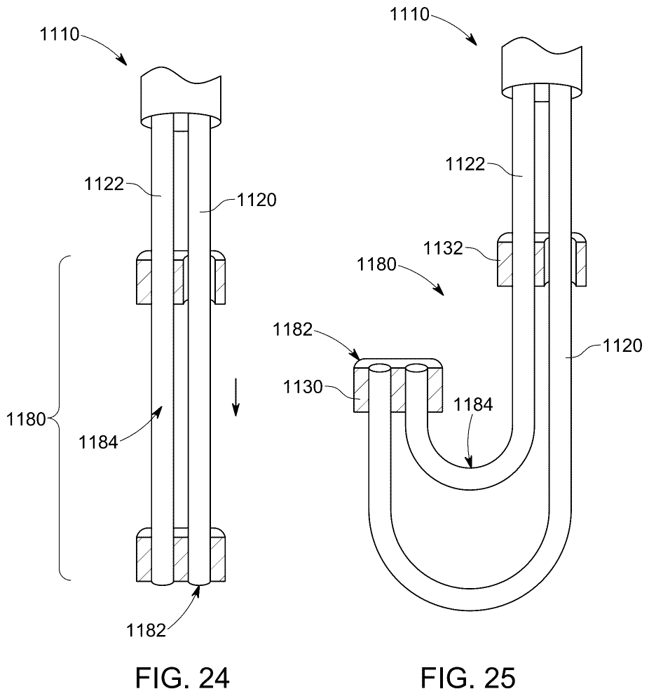

FIGS. 24 and 25 illustrate another embodiment of the surgical system 1110 with a cutting assembly 1180 used to traverse and lacerate a leaflet. The surgical system 1110 includes a catheter having a proximal end, a distal end opposite the proximal end, and a channel that extends from the distal end toward the proximal end. The surgical system 1110 also includes a first advancement shaft 1120 in the channel with the first shaft having a distal tip. A second advancement shaft 1122 in the channel is adjacent to the first advancement shaft but separate from the first advancement shaft. The second shaft having a distal tip. A coupler 1130 is fixed to the distal tips of the first advancement shaft 1120 and the second advancement shaft 1130. A guide member 1132 has a first component fixed to the second advancement shaft 1122 and a second component defining a guide channel that carries the first advancement shaft 1120. The first advancement shaft 1120 is movable within the guide channel.

The surgical system 1110 includes a cutting assembly 1180 with a first electrode positioned at the distal tip of the first advancement shaft 1120, and a second electrode 1184 located along a sidewall of the second advancement shaft 1122 at a location between the first component and the second component. Advancement of the first shaft in a distal direction causes a distal portion of the first and second shafts to form respective hook shapes such that the second electrode 1184 is positioned in an inner surface of a bend of the second shaft. In the illustrated embodiment, the second shaft separates between the first and second components from the first shaft due the coupler fixed to the distal tips of the first and second shafts. In the embodiment shown, the first shaft and the second shaft are electrically conductive and include an insulative coating. In this example, the first electrode is a denuded portion of the first shaft and the second electrode 1184 is a denuded portion of the second shaft. In another example, the first electrode is mounted on the distal tip of the first shaft, and the second electrode 1184 is mounted on the second shaft. Optionally, the leading surface of the first shaft may be positioned distal to the leading surface of the second shaft to optimize electrical contact between the distal cutting surface of the first shaft and the target leaflet while traversing the leaflet.

In use, a user can position a distal end of a catheter at a location proximate a leaflet of a valve so that the distal tip of the first and second advancement shaft contact the leaflet. The method may include electrifying a first electrode coupled to the distal tip of the first advancement shaft to traverse the leaflet. Next, a user may advance the cutting assembly 1180 through the leaflet. Next, a user may advance the first advancement shaft relative to the second advancement shaft so that the cutting assembly 1180 curves into a hook shape. In the hook shape, the second electrode is located on an inner surface of a bend of the hook shape shafts. The user can retract the catheter with the second advancement shaft in the form of the hook so that the second electrode lacerates the leaflet.

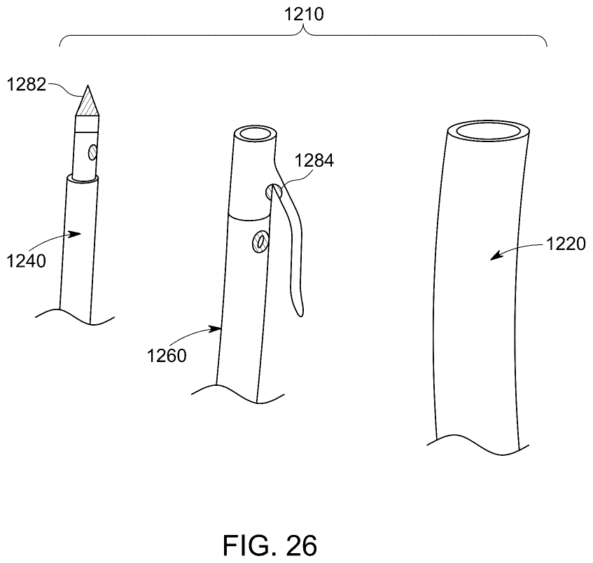

Another embodiment of the present disclosure is shown in FIG. 26 as a coaxial catheter system that can be used to traverse and lacerate the leaflet in sequence. The system may include a steerable catheter 1220 having a proximal end, a distal end opposite the proximal end, and an inner channel that extends from the distal end toward the proximal end. The distal end of the steerable catheter is bendable via an actuator or controller to direct the distal end to a desired orientation relative to the leaflet.

Continuing with FIG. 26, the system includes a first catheter 1240 insertable and movable in the inner channel of the steerable catheter 1220. The first catheter 1240 has a proximal end, a distal end opposite the proximal end, and an optional first channel that extends from the distal end toward the proximal end of the first catheter. The distal end of the first catheter 1240 having a distal tip including a first electrode 1282 that is coupled to a power source. The first catheter 1240 is insertable into the inner channel of the steerable catheter 1220 and movable such that the first electrode 1282 extends out of the distal end of the steerable catheter to contact the leaflet during use. The first catheter 1240 is configured to pierce or traverse the leaflet when the first electrode 1282 is electrified and in contact with the leaflet.

Continuing with FIG. 26, the system includes a second catheter 1260 movable over the first catheter 1240 and insertable and movable inside the channel of the steerable catheter 1220. Alternatively, in another embodiment, the first catheter can be removed from the steerable catheter 1220 prior to inserting the second catheter 1260 into the steerable catheter 1220. The second catheter 1260 has a proximal end, a distal end opposite the proximal end, and a second channel that extends from the distal end toward the proximal end of the second catheter. The distal end of the second catheter 1260 has a hook with a second electrode 1284 at an inner surface of the bend or along its outer surface and proximate the hook. The second catheter 1260 is insertable into and removable from the inner channel of the steerable catheter 1220. The second catheter 1260 is movable such that the hook and the second electrode 1284 extend out of the distal end of the steerable catheter 1220. The second electrode 1284 may be used to lacerate the leaflet after it is inserted through the opening formed by the first catheter 1240.

In use, a user may position a distal end of a steerable catheter into or near a valve, the steerable catheter having a proximal end opposite the distal end, and an inner channel that extends from the distal end toward the proximal end. The method may then include steering the distal end of the steerable catheter toward a target location of a leaflet of the valve. Next, a user may insert a distal end of a first catheter into and through the inner channel of the steerable catheter until a first electrode at the distal end of the first catheter is positioned adjacent to the leaflet. The user can electrify the first electrode to form an opening in the leaflet of the valve. With the first catheter traversed through the opening of the leaflet of the valve, the second catheter can be delivered over the first catheter. Alternatively, the user can remove the first catheter from the inner channel of the steerable catheter and insert the second catheter into the inner channel of the steerable catheter until its distal end extends through the opening of the leaflet of the valve. Next, a user can further advance the second catheter until the hook, having a second electrode on the inner surface of a bend, is positioned through the opening. The user can then retract the second catheter, while electrifying the second electrode to lacerate the leaflet of the valve.

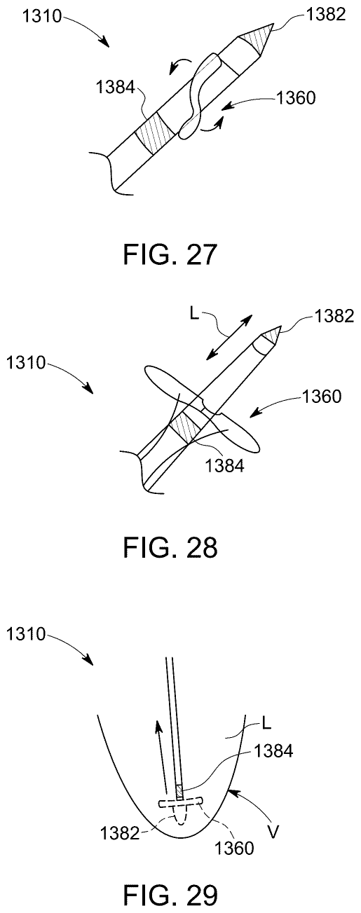

FIGS. 27-29 illustrate another embodiment of the surgical system 1310 used to traverse and lacerate a leaflet L of a valve V. The surgical system 1310 includes a shaft that is elongated along a longitudinal direction and has a proximal end, a distal end opposite the proximal end along and a channel that extends from the distal end toward the proximal end. The system 1310 includes a first electrode 1382 located at a distal-most tip of the shaft with the first electrode 1382 configured to form an opening in the leaflet. The system includes a movable anchor 1360 carried by the shaft and located proximal relative to the first electrode 1382. The movable anchor 1360 has a first end, a second end opposite the first end, a length that extends from the first end to the second end, and a coupler between the first and second ends that movably couples the anchor 1360 to the shaft. A second electrode 1384 on the shaft is located proximal relative to the movable anchor. The second electrode 1384 is configured to lacerate the leaflet. The movable anchor 1360 has an insertion configuration, as shown in FIG. 27, where the length of the anchor 1360 is aligned along the longitudinal direction L, and an anchoring configuration, as shown in FIGS. 28 and 29, where the anchor 1360 is rotated so that the length of the anchor is transverse to the longitudinal direction L. In operation, advancement of the movable anchor out a distal end of a catheter causes the movable anchor to transition from the insertion configuration into the anchoring configuration. Furthermore, when the movable anchor is in the anchoring configuration, the anchor is configured to secure the second electrode in position relative to the leaflet.

In use, a user may insert a distal end of a shaft into a heart proximate a leaflet of a valve. Then, the user can electrify a first electrode located at a distal-most tip of the distal end of the shaft in order to pierce an opening in the leaflet. The shaft may be advanced in a proximal direction until a movable anchor is through the opening. The user transitions the movable anchor from an insertion configuration, where a length of the anchor is aligned along a longitudinal direction, into an anchoring configuration where the anchor is rotated so that the length of the anchor is transverse to the longitudinal direction, in order to anchor the shaft in place relative to the leaflet. The user may retract the shaft with the anchor in the anchoring configuration to apply tension to the leaflet and electrify a second electrode on the shaft and located proximal relative to the movable anchor, in order to lacerate the leaflet.

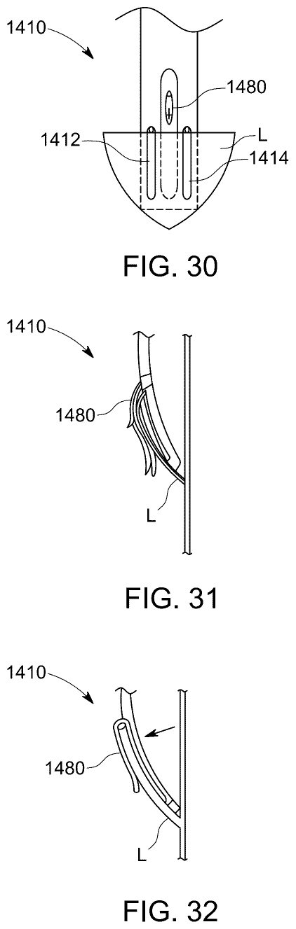

FIGS. 30-34 illustrate yet another embodiment a surgical system 1410 to lacerate a leaflet L. The surgical system 1410 may include a shaft that is elongated along a longitudinal direction. The shaft has a proximal end, a distal end opposite the proximal end along, a channel that extends from the distal end toward the proximal end, and a slot that extends from the distal end toward the proximal end. The surgical system may include a cutting assembly 1480 in the form of a hook extending from the slot of the shaft and aligned along an axis that is generally parallel to the longitudinal direction. The cutting hook is movable along the slot in the distal and proximal directions as needed.

As shown in FIGS. 30 and 31, the surgical system 1410 includes a first arm member 1412 that extends out from the shaft. The first arm member 1412 has a first bend and a first supporting arm that extends from the bend in a distal direction. A second arm member 1414 extends out from the shaft. The second arm member 1414 has a second bend and a second supporting arm that extends from the bend in the distal direction. As shown, the cutting hook is aligned along that axis that is generally between the first arm member 1412 and the second arm member 1414.

In use, the method includes inserting a distal end of shaft into a heart proximate to the base of a leaflet of a valve. Next, as shown in FIG. 32, while holding the leaflet in place with the first and second arm members, the user can cause the shaft to align with the leaflet, along its curvature. The user then advances a cutting hook in the slot of the shaft in a distal direction to lacerate the leaflet (as shown in FIG. 31).

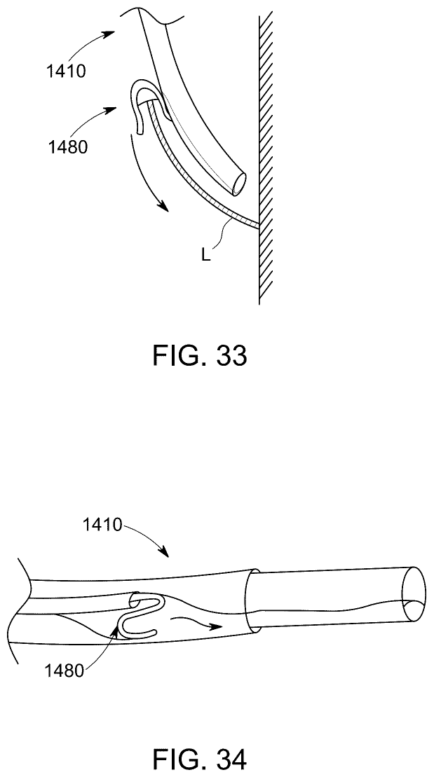

FIGS. 33 and 34 show an alternate version of the system shown in FIGS. 30 through 32. This version of the system functions the same but does not include supporting arms. The surgical system 1410 may include a shaft that is elongated along a longitudinal direction. The shaft has a proximal end, a distal end opposite the proximal end along, a channel that extends from the distal end toward the proximal end, and a slot that extends from the distal end toward the proximal end. The surgical system may include a cutting assembly 1480 in the form of a hook extending from the slot of the shaft and aligned along an axis that is generally parallel to the longitudinal direction. The cutting hook is movable along the slot in the distal and proximal directions as needed.

In use, the method includes inserting a distal end of shaft into a heart proximate to the base of a leaflet of a valve. Next, as shown in FIG. 33, while holding the leaflet in place, the user can cause the shaft to align with the leaflet, along its curvature. The user then advances a cutting hook in the slot of the shaft in a distal direction to lacerate the leaflet.

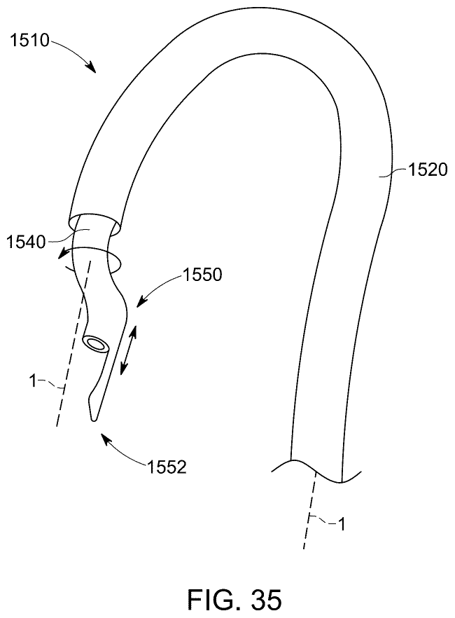

FIG. 35 includes another surgical system 1510 used in procedures for traversing, lacerating and/or excising a leaflet of a valve. The surgical system 1510 includes a steerable catheter 1520 having a proximal end, a distal end opposite the proximal end, and an inner channel that extends from the distal end toward the proximal end. The distal portion of the steerable catheter 1520 is bendable in one or more planes to accommodate the curvature of the aorta.

The system 1510 includes an inner catheter 1540 that is insertable and movable in the inner channel of the steerable catheter (coaxial with the steerable catheter). The inner catheter 1540 has a proximal end, a distal end opposite the proximal end, a distal portion 1550 that extends from the distal end toward the proximal end, and a channel that extends from the distal end toward the proximal end of the inner catheter.

The distal portion 1550 of the inner catheter may be pre-formed into a curved shape. In addition, the distal portion 1550 has a port that opens the channel, and a hood extension 1552 that extends in a distal direction from the port. The hood extends only partly around a central axis of the inner catheter. The hood serves as a positioning element, that may be positioned in the target leaflet nadir, to improve positioning and positional stability of a distal end of the catheter relative to the tissue, e.g. the target leaflet during a leaflet excision procedure. The positioning hood may be cut, molded, heat formed, or affixed to the inner catheter. The positioning hood may comprise a radiopaque material or added radiopaque feature. The positioning hood may flex outwardly slightly relative to the shaft 12 to aid in positioning. Furthermore, the inner catheter 1540 is rotatable about the central axis 1 in the inner channel of the steerable catheter. For instance, the inner catheter 1540 is rotatable 360 degrees about the central axis 1. In the embodiment shown, the distal portion 1550 of the inner catheter has an insertion configuration, when the distal portion 1550 is inside the inner channel and is generally linear or aligned inside the steerable catheter, and a steering configuration when the distal portion 1550 is outside of the inner channel. The distal portion 1550 in the steering configuration has a first portion that is generally aligned with a central axis 1 of the steerable catheter, and a second portion that is offset with respect to the central axis 1. The distal portion 1550 curves between the first portion and the second portion curves to form the offset. This offset and pre-formed shape is configured to provide access to and proper angle to a nadir of a leaflet of a valve. For instance, the curved distal portion of the inner catheter may be advanced distally, relative to the distal end of the steerable catheter, and directed into the nadir of a leaflet. Additional surgical devices include cutting assemblies as described herein may be inserted through the channel of the inner catheter to position the cutting assemblies at the desired location relative to the leaflet.

For instance, in all cases where there is a cutting assembly with traversing cutters, slitting cutters or c-shaped cutter, and/or one or more hook cutters, such components and systems may be used in combination with the steerable catheter system as shown in FIG. 35 and described above.

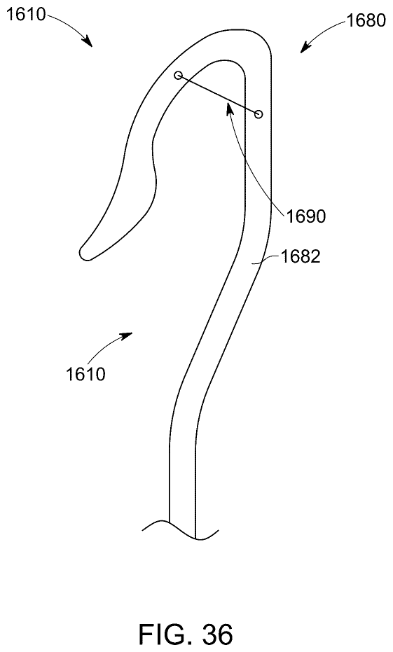

FIG. 36 includes yet another embodiment of a surgical system 1610 used to lacerate and/or excise a leaflet of a valve. As shown, the surgical system 1610 includes a shaft that is elongated along a central axis. The shaft has a proximal end, and a distal end opposite the proximal end along the central axis. A cutting assembly 1680 includes a hook assembly at the distal end of the shaft and includes a hook 1682. The hook has a shank, a bend, and a tip extending from the bend, the tip and the shank defining a gape therebetween. An electrode wire 1690 extends from the tip to the shank across the gape of the hook 1682. In one example, the electrode wire 1690 is a separate conductor attached to the hook 1682. In addition, the hook 1682 itself can be conductive and conducts current to the electrode wire 1690. The one hook embodiments shown here and described elsewhere in the patent application is configured for lacerating a leaflet. However, this particular embodiment, the surgical system 1610, may include a second hook (not shown) with a second electrode wire that spans the gape of the second hook. Two hook systems are used for excising a leaflet. The surgical system described here may be used in combination with the surgical system and components described in U.S. application Ser. No. 18/127,428 and International Application No. PCT/US2023/016571, the entire contents of which are incorporated by reference herein. In one example, the hook body (shank, bend and tip) is coated to insulate the hook body and at least a portion of the electrode wire is not coated or denuded to focus the electrical energy to at least a portion of the wire.

In use, a user may insert a distal end of a shaft proximate a first side a leaflet of a valve. The user can position a hook carried by the shaft so that the hook is on a second side opposite of the first side of the leaflet of the valve. The user can electrify the electrode wire that extends across the gape of the hook. Then, a user can retract the shaft, while the electrode wire is electrified, to lacerate the leaflet of the valve. In alternative embodiments where a second hook is also utilized, the user can excise the leaflet of the valve. Alternatively, this hook embodiment can be movable within a shaft to lacerate a leaflet as described above.

While the electrode wire 1690 is specifically described in the embodiment encompassing FIG. 36, it should be noted that the electrode wire 1690 may be utilized in any embodiment involving single hooks for lacerating a leaflet as well as embodiments involving a first hook and a second hook for excising the leaflet.

The surgical systems as described herein include one or more of the elongated shafts, which could be in the form of catheters. The shafts described herein, when in the form of catheters, will generally include a shaft, an inner channel, one or more radiopaque markers, and a distal tip. One of or more catheters as described herein may have a secondary curve, a primary curve, or no pre-set curves. The primary and secondary curves are not illustrated in the drawings. The distal tip defines the distal most end of each elongated shaft. The shaft may, in an alternate embodiment, include an inner channel that is also sized to receive other surgical devices therethrough. For example, the surgical system 10 may be, but is not required to, receive a guidewire such that an over-the-wire technique may be used. That is, a guidewire can be placed through the valve structure into the left ventricle and the distal end of the surgical system or separate steering catheter is inserted over the guidewire into position. In an alternative embodiment, the surgical system or separate steering catheter, or one or more of its shafts, may include one or more skive ports that can be used to receive the guidewire therethrough. Such skive ports may be disposed toward or along an outer surface of the shaft. In yet another embodiment, the guidewire may not extend through the valve structure into the ventricle. The surgical system, however, may still slide over or along the guidewire, but without the benefit of having the guidewire cross through the valve structure.

In cross-section, a catheter may include an inner liner, a middle reinforcing layer (e.g. a braid), and an outer layer or outer jacket. In addition, the catheter may be a biaxial design that includes an additional outer layer to minimize interaction with the introducer and/or sheath and allow smoother movement of the surgical system. In another embodiment, the catheter would also be able to accommodate different shaped inner catheters to achieve a suitable relationship of the distal catheter tip to the leaflet. For example, this configuration may provide for functionality similar to the use of a 5 F/6 F 120 mm IM catheter inside an AL type catheter, i.e., a mother and daughter technique. The catheter may be configured to transition in response to operator input to assume different degrees of flexion of the distal tip to account for different patient anatomy.

The longitudinal shape of the catheter can vary as needed. For instance, the catheter can have a shape according to the Amplatz Guide that includes, but is not limited to AL-1, AL-2, AL-3, AL-4, etc. Other common shapes are possible as well. In one example, the catheter may have an outer cross-sectional dimension sized for insertion into the aorta. For instance, the catheter may be either 12 French or 14 French. However, larger, or smaller sized catheters may be used in certain instances. The catheter tip or distal tip may be deflectable or bendable as needed to steer the distal tip into position, for example, when using a steering element as described above. The catheter may also be configured to accommodate different shaped inner catheters.

The catheter has at least one port that extends to the inner channel. As shown, the at least one port could be two or more as needed. The port or ports are spaced a distance from the leading end that is less than a distance between the at least one port and the trailing end. In other words, they are positioned toward the leading end of the catheter. These ports are intended to a) allow for flushing or priming the system prior to introduction to the patient and/or b) allow removal of emboli, such as air and other debris after cutting, and throughout, to provide for hemodynamic monitoring of the blood pressure in the ascending aorta. The ports may also be used for contrast injection, as needed. For instance, when the leaflets get cut, the destruction of the aortic valve may lead to decompensation of coronary output, which is monitored by a local lumen. The system may, in turn, include a luer fitting on the handle for monitoring and bubble removal. Bubble and debris removal can happen via an active ‘vac lock’ syringe (pull a vacuum with a syringe and the handle locks in place so holding by the user is not required) on the port for evacuating 50-100 ml of blood/air.

In all cases, the surgical system may be used with a native valve, an implanted artificial valve, or a valve that is implanted on a surgical ring, or a valve-in-valve.

It will be appreciated by those skilled in the art that various modifications and alterations of the present disclosure can be made without departing from the broad scope of the appended claims. Some of these have been discussed above and others will be apparent to those skilled in the art. The scope of the present disclosure is limited only by the claims.

1. A surgical system, comprising:
a shaft having a proximal end, a distal end opposite the proximal end, and a channel that extends from the distal end toward the proximal end;
a slit cutter configured to form a slit in a tissue and being movable in the channel, the slit cutter including a distal cutting surface; and
a hook in the channel, the hook having a shank, a bend, and a tip, wherein the hook includes an inner cutting surface at an inner surface of the bend, such that the distal cutting surface and the inner cutting surface are oriented in generally opposite directions,
wherein the slit cutter and the hook are independently movable relative to each other.

2. The surgical system according to claim 1, wherein the shaft is a catheter.

3. The surgical system according to claim 1, wherein the slit cutter is electrically insulated and includes a denuded portion at the distal cutting surface.

4. The surgical system according to claim 1, wherein the hook is electrically insulated and includes a denuded portion at the inner cutting surface.

5. The surgical system according to claim 1, wherein the slit cutter includes a planar body having a first planar surface, a second planar surface opposite the first planar surface, and a sidewall that extends between the first planar surface and the second planar surface, wherein a distal-most portion of the sidewall defines the distal cutting surface.

6. The surgical system according to claim 1, wherein the slit cutter includes a wall that extends partially around a central axis to form a guide channel, the wall having an outer surface, an inner surface, and the distal cutting surface, wherein the hook is nested within the guide channel.

7. The surgical system according to claim 6, wherein the distal cutting surface is denuded.

8. The surgical system according to claim 6, wherein a portion of the slit cutter is tapered.

9. The surgical system according to any one of the claims 1 to 7, wherein the slit cutter and the hook are electrically isolated from each other.

10. The surgical system according to any one of the claims 1 to 7, wherein the slit cutter and the hook are independently coupled to electric power.

11. A method, comprising:
positioning a distal end of a shaft toward a leaflet of a valve, the shaft having a channel, a slit cutter and a hook each located in the channel;
electrifying a slit cutter carried in a channel of the shaft;
advancing the slit cutter and a hook through the leaflet so that the slit cutter forms a slit in the leaflet;
retracting the slit cutter relative to the hook;
engaging the hook with the leaflet;
electrifying the hook; and
retracting the hook to lacerate the leaflet.

12. The method according to claim 11, using the surgical system according to anyone of claims 1 to 10.

13. A surgical system, comprising:
a shaft having a proximal end, a distal end opposite the proximal end, and a channel that extends from the distal end toward the proximal end;
a first hook in the channel, the first hook having a shank, a bend, and a distal cutting surface at an outer portion of the bend; and
a second hook in the channel and configured to move with the first hook in the channel, the second hook having a shank, a bend, and an inner cutting surface at an inner portion of the bend, such that the distal cutting surface and the inner cutting surface are oriented in generally opposite directions,
wherein the first and second hooks are movable together relative to the shaft.

14. The surgical system according to claim 13, wherein the shaft is a catheter.

15. The surgical system according to any one of the claims 13 to 14, wherein the first and second hooks are electrically isolated from each other.

16. The surgical system according to any one of the claims 13 to 15, wherein the first and second hooks are independently coupled to electric power.

17. The surgical system according to claim 13, wherein the first hook and the second hook are insulated and include a first denuded portion at the distal cutting surface and a second denuded portion at the inner cutting surface.

18. The surgical system according to claim 13, wherein the first hook is configured to form a slit in a leaflet of a valve, and the second hook is configured to lacerate the leaflet of the valve.

19. The surgical system according to claim 13, wherein movement of the first hook and the second hook in a first direction cause the first hook to form a slit in a leaflet of a valve, and movement of the first hook and the second hook in the second direction that is opposite the first direction cause the second hook to lacerate the leaflet of the valve.

20. The surgical system according to claim 13, wherein the first hook and the second hook are attached to each other.

21. The surgical system according to claim 13, wherein the first hook does not have a tip and the second hook has a tip.

22. The surgical system according to claim 13, wherein the first hook is positioned distal relative to the second hook.

23. The surgical system according to claim 13, wherein the second hook is nested in the bend of the first hook.

24. A method, comprising:
positioning a distal end of a shaft toward a leaflet of a valve, the shaft having a channel, a first hook and a second hook attached to the first hook;
electrifying a distal cutting surface of the first hook to form a slit in a leaflet of a valve;
advancing the first hook and a second hook through the slit in the leaflet in a distal direction;
engaging the first and second hooks with the leaflet;
electrifying an inner cutting surface of the second hook; and
retracting the first and second hooks in a proximal direction that is opposite the distal direction to lacerate the leaflet with the inner cutting surface of the second hook.

25. A method according to claim 24, using the surgical system according to claim any one of the claims 13 to 23.

26. A surgical system, comprising:
a shaft having a proximal end and a distal end opposite the proximal end, and a channel that extends from the distal end toward the proximal end; and
a hook carried by the shaft, the hook having a shank, a bend, and a distal electrode at an outer portion of the bend, and an inner electrode at an inner surface of the bend, wherein the hook is configured to cut tissue when advancing in both a distal direction and a proximal direction.

27. The surgical system according to claim 26, wherein the shaft is a catheter.

28. A method using the surgical system according to any one of claims 26 and 27.

29. A surgical system, comprising:
a shaft having a proximal end, a distal end opposite the proximal end, and a channel that extends from the distal end toward the proximal end;
a first electrode located at a distal-most end of the distal end of the shaft;
a second electrode located along a sidewall of the shaft at a location that is proximal relative to the first electrode; and
an internal pull wire in the shaft having a distal end that is coupled to the distal end of the shaft, the internal pull wire configured to, upon application of tension to the internal pull wire, cause the distal end of the shaft to curve into a hook such that the second electrode is positioned on an inner surface of a bend in the hook,
wherein the first electrode is configured to traverse a leaflet of a valve and the second electrode is configured to lacerate the leaflet.

30. The surgical system according to claim 29, wherein the shaft is a catheter.

31. The surgical system according to claim 29 or claim 30, wherein the catheter has a traverse configuration where the catheter is generally linear shaped, and a lacerating configuration wherein the distal end of the catheter is in the form of the hook.

32. The surgical system according to any one of the claims 29 to 31, wherein the first and second electrodes are electrically isolated from each other.

33. The surgical system according to any one of the claims 29 to 32, wherein the first and second electrode are independently coupled to electric power.

34. The surgical system according to any one of the claims 29 to 33, further comprising a steerable catheter having a channel extending therethrough, wherein the catheter is movable in the channel to permit the distal end to extend out of a distal end of the steerable catheter.

35. A method using the surgical system according to claims 29 to 34.

36. A surgical system, comprising:
a catheter having a proximal end, a distal end opposite the proximal end, and a channel that extends from the distal end toward the proximal end;
a first advancement shaft in the channel, the first shaft having a distal tip;
a second advancement shaft in the channel adjacent to the first advancement shaft, the second shaft having a distal tip;
a coupler fixed to the distal tips of the first shaft and the second shaft;
a guide member having a first component fixed to the second shaft and having a second component defining a guide channel that carries the first shaft, wherein the first shaft is movable within the guide channel;
a first electrode positioned at the distal tip of one or both of the first and second shafts; and
a second electrode position on the second shaft at a location between the first component and the second component,
wherein the advancement of the first shaft in a distal direction causes a distal portion of the first and second shafts to form respective hook shapes such that the second electrode is positioned in an inner surface of a bend of the second shaft.

37. The surgical system according to claim 36, wherein the first shaft and the second shaft are electrically conductive and include an insulative coating, wherein the first electrode is a denuded portion of the first shaft and the second electrode is a denuded portion of the second shaft.

38. The surgical system according to claim 36 or claim 37, wherein the first electrode is mounted on the either or both of the distal tips of the first and second shafts, and the second electrode is mounted on the second shaft.

39. The surgical system according to any one of the claims 36 to 38, wherein a distal portion of the catheter is steerable.

40. A method, comprising:
inserting a catheter to a location proximate a leaflet of a valve so that distal tip of the first and second advancement shafts contact the leaflet;
positioning a distal end of a catheter toward a leaflet of a valve, the catheter having a channel, a first advancement shaft, a second advancement shaft separate from the first advancement shaft, wherein a coupler fixes a distal tip of the first advancement shaft to the distal tip of the second advancement shaft;
electrifying a first electrode coupled to the distal tip of the first advancement shaft to traverse the leaflet;
advancing the first advancement shaft relative to the second advancement shaft so that the second advancement shaft curves into a hook, wherein the hook has a second electrode on an inner surface of a bend of the hook; and
retracting the catheter with the second advancement shaft in the form of the hook so that the second electrode lacerates the leaflet.

41. The method of claim 36, using the surgical system according to anyone of the claims 36 to

42. A surgical system, comprising:
a steerable catheter having a proximal end, a distal end opposite the proximal end, and an inner channel that extends from the distal end toward the proximal end, wherein the distal end of the steerable catheter is bendable via a controller to direct the distal end to a desired orientation;
a first catheter insertable and movable in the first channel of the steerable catheter, the first catheter having a proximal end, a distal end opposite the proximal end, and an optional first channel that extends from the distal end toward the proximal end of the first catheter, the distal end of the first catheter having a distal tip including a first electrode, wherein the first catheter is insertable into and removable from the inner channel of the steerable catheter; and
a second catheter movable over the first catheter, the second catheter having a proximal end, a distal end opposite the proximal end, and a second channel that extends from the distal end toward the proximal end of the second catheter, the distal end of the second catheter having a hook with second electrode at an inner surface of the bend or along an outer surface of the second catheter proximate the hook, wherein the second catheter is insertable into and removable from the inner channel of the steerable catheter.

43. The surgical system according to claim 42, wherein the first catheter is insertable into the inner channel of the steerable catheter and movable such that the first electrode extends out of the distal end of the steerable catheter,
wherein the second catheter is insertable into the inner channel of the steerable catheter and movable such that the hook and the second electrode extend out of the distal end of the steerable catheter.

44. A method, comprising:
inserting a distal end of a steerable catheter into or near a valve, the steerable catheter having a proximal end opposite the distal end, and an inner channel that extends from the distal end toward the proximal end;
steering the distal end of the steerable catheter toward a target location of a leaflet of the valve;
inserting a distal end of a first catheter into and through the inner channel of the steerable catheter until a first electrode at the distal end of the first catheter is positioned adjacent to the leaflet;
electrifying a first electrode to form an opening in the leaflet of the valve;
optionally removing the first catheter from the inner channel of the steerable catheter;
inserting the distal end of a second catheter into the inner channel of the steerable catheter until the distal end extends through the opening of the leaflet of the valve;
causing a hook to advance from the distal end of the second catheter until a hook having a second electrode on the inner surface of a bend of the hook is positioned through the opening; and
retracting the second catheter, while electrifying the second electrode to lacerate the leaflet of the valve.

45. The method according to claim 44, using the surgical system according to any one of the claims 42-43.

46. A surgical system, comprising:
a shaft that is elongated along a longitudinal direction, the shaft having a proximal end, a distal end opposite the proximal end along and a channel that extends from the distal end toward the proximal end;
a first electrode located at a distal-most tip of the shaft, the first electrode configured to form an opening in tissue;
a movable anchor carried by the shaft and located proximal relative to the first electrode, the movable anchor having a first end, a second end opposite the first end, a length that extends from the first end to the second end, and a coupler between the first and second ends that movably couple the anchor to the shaft; and
a second electrode on the shaft and located proximal relative to the movable anchor, wherein the second electrode is configured to lacerate the tissue;
wherein the movable anchor has an insertion configuration where the length of the anchor is aligned along the longitudinal direction, and an anchoring configuration where the anchor is rotated so that the length of the anchor is transverse to the longitudinal direction.

47. The surgical system according to claim 46, wherein advancement or rotation of the movable anchor out of a distal end of a catheter causes the movable anchor to transition from the insertion configuration into the anchoring configuration.

48. The surgical system according to claim 46, wherein when the movable anchor is in the anchoring configuration, the movable anchor is configured to secure the second electrode in position relative to the tissue.

49. A method, comprising:
inserting a distal end of a shaft into a heart proximate a leaflet of a valve;
electrifying a first electrode located at the distal-most tip of the distal end of the shaft in order to pierce an opening in the leaflet;
advancing the shaft in a proximal direction until a movable anchor is through the opening;
transitioning the movable anchor from an insertion configuration where a length of the anchor is aligned along a longitudinal direction, into an anchoring configuration where the anchor is rotated so that the length of the anchor is transverse to the longitudinal direction, in order to anchor the shaft in place relative to the leaflet;
retracting the shaft with the anchor in the anchoring configuration to apply tension to the leaflet; and
electrifying a second electrode on the shaft and located proximal relative to the movable anchor, in order to lacerate the leaflet.

50. The method of claim 46 using the surgical system according to anyone of the claims 43 to 44.

51. A surgical system, comprising:
a shaft that is elongated along a longitudinal direction, the shaft having a proximal end, a distal end opposite the proximal end, a channel that extends from the distal end toward the proximal end, and a slot that extends from the distal end toward the proximal end; and
a cutting hook extending from the slot of the shaft and aligned along an axis that is generally parallel to the longitudinal direction, wherein the cutting hook is movable along the slot and within the channel and is configured to lacerate a leaflet.

52. The surgical system according to claim 51, further comprising:
a first arm member that extends out from the shaft, the first arm member having a first bend and a first supporting arm that extends from the bend in a distal direction; and
a second arm member that extends out from the shaft, the second arm member having a second bend and a second supporting arm that extends from the bend in the distal direction,
wherein the first arm and the second arm are configured to secure the leaflet to the shaft and wherein the cutting hook is aligned along the axis that is generally between the first arm member and the second arm member.

53. A method, comprising:
inserting a distal end of a shaft into a heart proximate to a base of a leaflet of a valve;
holding the leaflet in place, causing the shaft to align with the leaflet; and
advancing a cutting hook that extends from a slot of the shaft in a distal direction to lacerate the leaflet.

54. The method of claim 53, using the surgical system according to claim 51 or claim 52.

55. A surgical system, comprising:
a steerable catheter having a proximal end, a distal end opposite the proximal end, and an inner channel that extends from the distal end toward the proximal end, wherein the distal end of the steerable catheter is bendable to direct the distal end to a desired orientation; and
an inner catheter insertable in the inner channel of the steerable catheter and movable in the inner channel relative to the steerable catheter, the inner catheter having a proximal end, a distal end opposite the proximal end, a distal portion that extends from the distal end toward the proximal end, and a channel that extends from the distal end toward the proximal end of the inner catheter, the distal portion of the inner catheter being pre-formed into a curved shape, the distal portion having a port that opens the channel, wherein the inner catheter is rotatable about the central axis in the inner channel of the steerable catheter.

56. The surgical system according to claim 55, wherein the inner catheter is rotatable 360 degrees about the central axis.

57. The surgical system according to claim 55, wherein the distal portion of the inner catheter is steerable.

58. The surgical system according to claim 55, wherein the distal portion of the inner catheter has an insertion configuration when the distal portion is inside the inner channel and a steering configuration when the distal portion is outside of the inner channel.

59. The surgical system according to claim 58, wherein the distal portion in the steering configuration has a first portion that is generally aligned with a central axis of the steerable catheter, and a second portion that is offset with respect to the central axis, wherein the distal portion curves between the first portion and the second portion to form the offset.

60. The surgical system according to claim 55, wherein distal portion is pre-formed to provide access to and proper angle to a nadir of leaflet of a valve.

61. A method for accessing a leaflet in a valve using the surgical system according to any one of claims 55 to 60.

62. A surgical system, comprising:
a shaft that is elongated along a central axis, the shaft having a proximal end, and a distal end opposite the proximal end along the central axis; and
a hook assembly carried by the distal end of the shaft, the hook assembly having one or more hooks, each of the one or more hooks having a shank, a bend, and a tip extending from the bend, wherein the tip has a narrow portion and lobe that defines a terminal end of the tip, wherein the tip is angled with respect to the distal end of the shank.

63. The surgical system according to claim 62, wherein the one or more hooks is a first hook and a second hook.

64. The surgical system according to claim 63, wherein the first hook and the second hook are mirror images of each other.

65. The surgical system according to claim 63, wherein the tip of the first hook and the second hook is not flexible.

66. The surgical system according to claim 63, wherein the bend and the tip of each hook is flexible.

67. The surgical system according to claim 62, wherein the tip of the one or more hooks includes an elongated portion that extends in a proximal direction and curves in a direction away from the shank.

68. The surgical system according to claim 62, wherein the bend of the one or more hooks includes one or more apertures.

69. The surgical system according to claim 62, wherein an inner surface of the bend of the one or more hooks is or includes an electrode.

70. The surgical system according to claim 62, wherein each hook includes a high strain zone and a high heat zone that is separate from the high strain zone.

71. A method for cutting tissue in a valve using the surgical system according to any one of the claims 62 to 70.

72. A surgical system, comprising:
a catheter having a proximal end, a distal end opposite the proximal end along a central axis, an inner surface, and an inner channel defined by the inner surface, the inner channel extending from the distal end toward the proximal end;
a cutting element fixed to the distal end of the catheter, wherein the cutting element extends around a portion of the central axis of the catheter; and
a cartridge in the inner channel and keyed to the inner surface of the catheter;
a hook assembly fixed to the cartridge, the hook assembly having a first hook and a second hook, wherein the hook assembly and cartridge are movable along a longitudinal direction but are not rotatable in the inner channel.

73. The surgical system according to claim 72, wherein the hook assembly has a first hook and a second hook.

74. The surgical system according to claim 72, wherein the cutting element is a C-shaped cutter.

75. The surgical system according to claim 72, wherein the inner surface of the catheter has at least one flat portion, wherein the cartridge has at least one flat side that faces the at least one flat portion.

76. The surgical system according to claim 72, further comprising an inner shaft having a distal end coupled to the cartridge, the inner shaft configured to cause the cartridge to move in a distal direction or a proximal direction to advance the hook assembly relative to the cutting element and retract the hook assembly relative to the cutting element, respectively.

77. A surgical system, comprising:
a shaft that is elongated along a central axis, the shaft having a proximal end, and a distal end opposite the proximal end along the central axis;
a hook assembly at the distal end of the shaft, the hook assembly having a hook with a shank, a bend, and a tip extending from the bend, the tip and the shank defining a gape therebetween; and
an electrode wire that extends from the tip to the shank across the gape of the hook.

78. The surgical system according to claim 80, wherein the electrode wire is a separate conductor attached to the hook.

79. The surgical system according to claim 80, wherein the hook is conductive and conducts current to the electrode wire.

80. The surgical system according to claim 80, wherein the hook is a first hook, the electrode wire is a first electrode wire, wherein the hook assembly includes a second hook and a second electrode wire.

81. The surgical system according to claim 80, wherein at least a portion of the electrode wire is uncoated and the hook is coated to electrically insulate the hook.

82. A method, comprising:
inserting a distal end of a shaft proximate a first side of a leaflet of a valve;
positioning a hook carried by the shaft so the hook is on a second side opposite of the first side of the leaflet of the valve;
electrifying an electrode wire that extends from a tip of the hook to a shank of the hook across a gape of the hook; and
retracting the shaft, while the electrode wire is electrified, to lacerate the leaflet of the valve.

83. A method according to claim 82 using the surgical system of anyone of the claims 81 to 86.

84. The surgical system of claim 7, wherein the denuded distal cutting surface includes one or more electrodes attached to the surface.

85. The surgical system of claim 17, wherein the first denuded portion includes one or more electrodes attached to the distal cutting surface and the second denuded portion includes one or more electrodes attached to the inner cutting surface.

86. The surgical system according to claim 23, wherein the first hook has a first engagement member that engages with a second engagement member of the second hook.

87. The surgical system according to claim 36, wherein the first electrode and the second electrode are denuded surfaces on the first and second shafts.

88. The surgical system according to claim 36, wherein the first electrode and the second electrode are attached electrodes.

89. The surgical system according to claim 46, further comprising an actuator that extends along a length of the shaft, the actuator configured to transition the moveable anchor from the insertion configuration to the anchoring configuration.

90. The surgical system according to claim 55, further comprising a hood extension that extends in a distal direction from the port, the hood extending only partly around a central axis of the inner catheter.

91. The surgical system according to claim 62, wherein the shank has a proximal end and a distal end, and a curved portion between the proximal end of the shank and the distal end of the shank.

92. The surgical system according to claim 62, wherein the bend and the tip curve around a part of the central axis of the shaft.
